SEQUENCE LISTING (173 sequences)

SEQ ID NO: 1 - DNA, 1302 bp, Homo sapiens
  source 1..1302: mol_type = genomic DNA
atgagggccgagggcctcggcggcctggagcgcttctgcagcccgggcaaaggccgggggctgcgggctctgcagcccttccaggtgggggacttgctgttctcctgcccggcctatgcctacgtgctcacggtcaacgagcggggcaaccactgcgagtactgcttcaccaggaaagaaggattgtccaaatgtggaagatgcaagcaggcattttactgcaatgtggagtgtcagaaagaagattggcccatgcacaagctggaatgttctcccatggttgtttttggggaaaactggaatccctcggagactgtaagactaacagcaaggattctggccaaacagaaaatccacccagagagaacaccttcggaaaaattgttagctgtgaaggagtttgaatcacatctggataagttagacaatgagaagaaggatttgattcagagtgacatagctgctctccatcacttttactccaagcatctcggattccctgacaatgatagcctcgtagtactctttgcacaggttaactgtaatggcttcacaattgaagatgaagaactttctcatttgggatcagcgatatttcctgatgttgcattgatgaatcatagctgttgccccaatgtcattgtgacctacaaagggaccctggcagaagtcagagctgtacaggaaatcaagccgggagaggaggtttttaccagctatattgatctcctgtacccaacggaagatagaaatgaccggttaagagattcttatttctttacctgtgagtgccaggagtgtaccaccaaggacaaggataaggccaaggtggaaatccggaagctcagcgatcccccaaaggcagaagccatccgagacatggtcagatatgcacgcaacgtcattgaagagttccggagggccaagcactataaatcccctagtgagctgctggagatctgcgagctcagccaggagaagatgagctctgtgtttgaggacagtaacgtgtacatgttgcacatgatgtaccaggccatgggtgtctgcttgtacatgcaggactgggaaggagccctgcaatatggacagaaaatcattaagccctacagtaagcactatcctttgtactccctcaacgtggcctccatgtggttgaagctagggagactctacatgggcctggaacacaaagccgcaggggagaaagccctgaagaaggccattgcaatcatggaagtagctcacggcaaagatcatccatatatttctgagatcaaacaggaaattgaaagccactga

SEQ ID NO: 2 - DNA, 23 bp, synthetic construct
  source 1..23: mol_type = other DNA
gaccggttaagagattcttattt

SEQ ID NO: 3 - DNA, 23 bp, synthetic construct
  source 1..23: mol_type = other DNA
cacggcaaagatcatccatatat

SEQ ID NO: 4 - DNA, 23 bp, synthetic construct
  source 1..23: mol_type = other DNA
tacccaacggaagatagaaatga

SEQ ID NO: 5 - DNA, 23 bp, synthetic construct
  source 1..23: mol_type = other DNA
gccctgcaatatggacagaaaat

SEQ ID NO: 6 - DNA, 23 bp, synthetic construct
  source 1..23: mol_type = other DNA
tcggagactgtaagactaacagc

SEQ ID NO: 7 - DNA, 23 bp, synthetic construct
  source 1..23: mol_type = other DNA
cagagctgtacaggaaatcaagc

SEQ ID NO: 8 - DNA, 23 bp, synthetic construct
  source 1..23: mol_type = other DNA
agccctgcaatatggacagaaaa

SEQ ID NO: 9 - DNA, 23 bp, synthetic construct
  source 1..23: mol_type = other DNA
ctcggattccctgacaatgatag

SEQ ID NO: 10 - DNA, 23 bp, synthetic construct
  source 1..23: mol_type = other DNA
atcagcgatatttcctgatgttg

SEQ ID NO: 11 - DNA, 23 bp, synthetic construct
  source 1..23: mol_type = other DNA
tgcacgcaacgtcattgaagagt

SEQ ID NO: 12 - DNA, 23 bp, synthetic construct
  source 1..23: mol_type = other DNA
ggcaaagatcatccatatatttc

SEQ ID NO: 13 - DNA, 23 bp, synthetic construct
  source 1..23: mol_type = other DNA
gaggtttttaccagctatattga

SEQ ID NO: 14 - DNA, 23 bp, synthetic construct
  source 1..23: mol_type = other DNA
ccggttaagagattcttatttct

SEQ ID NO: 15 - DNA, 23 bp, synthetic construct
  source 1..23: mol_type = other DNA
gagatcaaacaggaaattgaaag

SEQ ID NO: 16 - DNA, 23 bp, synthetic construct
  source 1..23: mol_type = other DNA
tggataagttagacaatgagaag

SEQ ID NO: 17 - DNA, 23 bp, synthetic construct
  source 1..23: mol_type = other DNA
tgcaatatggacagaaaatcatt

SEQ ID NO: 18 - DNA, 23 bp, synthetic construct
  source 1..23: mol_type = other DNA
accggttaagagattcttatttc

SEQ ID NO: 19 - DNA, 23 bp, synthetic construct
  source 1..23: mol_type = other DNA
gagattcttatttctttacctgt

SEQ ID NO: 20 - DNA, 23 bp, synthetic construct
  source 1..23: mol_type = other DNA
cggcaaagatcatccatatattt

SEQ ID NO: 21 - DNA, 23 bp, synthetic construct
  source 1..23: mol_type = other DNA
tagaaatgaccggttaagagatt

SEQ ID NO: 22 - DNA, 23 bp, synthetic construct
  source 1..23: mol_type = other DNA
gtgaaggagtttgaatcacatct

SEQ ID NO: 23 - DNA, 23 bp, synthetic construct
  source 1..23: mol_type = other DNA
ccctcggagactgtaagactaac

SEQ ID NO: 24 - DNA, 23 bp, synthetic construct
  source 1..23: mol_type = other DNA
ctctgtgtttgaggacagtaacg

SEQ ID NO: 25 - DNA, 23 bp, synthetic construct
  source 1..23: mol_type = other DNA
taccagctatattgatctcctgt

SEQ ID NO: 26 - DNA, 23 bp, synthetic construct
  source 1..23: mol_type = other DNA
tggcttcacaattgaagatgaag

SEQ ID NO: 27 - DNA, 23 bp, synthetic construct
  source 1..23: mol_type = other DNA
ttcacaattgaagatgaagaact

SEQ ID NO: 28 - DNA, 23 bp, synthetic construct
  source 1..23: mol_type = other DNA
ggcttcacaattgaagatgaaga

SEQ ID NO: 29 - DNA, 23 bp, synthetic construct
  source 1..23: mol_type = other DNA
tcctgtacccaacggaagataga

SEQ ID NO: 30 - DNA, 23 bp, synthetic construct
  source 1..23: mol_type = other DNA
gggtgtctgcttgtacatgcagg

SEQ ID NO: 31 - DNA, 23 bp, synthetic construct
  source 1..23: mol_type = other DNA
ctgcaatgtggagtgtcagaaag

SEQ ID NO: 32 - DNA, 23 bp, synthetic construct
  source 1..23: mol_type = other DNA
cacgcaacgtcattgaagagttc

SEQ ID NO: 33 - DNA, 23 bp, synthetic construct
  source 1..23: mol_type = other DNA
ccctgacaatgatagcctcgtag

SEQ ID NO: 34 - DNA, 23 bp, synthetic construct
  source 1..23: mol_type = other DNA
gccccaatgtcattgtgacctac

SEQ ID NO: 35 - DNA, 23 bp, synthetic construct
  source 1..23: mol_type = other DNA
aggagaagatgagctctgtgttt

SEQ ID NO: 36 - DNA, 23 bp, synthetic construct
  source 1..23: mol_type = other DNA
atcatccatatatttctgagatc

SEQ ID NO: 37 - DNA, 23 bp, synthetic construct
  source 1..23: mol_type = other DNA
atccatatatttctgagatcaaa

SEQ ID NO: 38 - DNA, 23 bp, synthetic construct
  source 1..23: mol_type = other DNA
ttgcattgatgaatcatagctgt

SEQ ID NO: 39 - DNA, 23 bp, synthetic construct
  source 1..23: mol_type = other DNA
gagactgtaagactaacagcaag

SEQ ID NO: 40 - DNA, 23 bp, synthetic construct
  source 1..23: mol_type = other DNA
ttgattcagagtgacatagctgc

SEQ ID NO: 41 - DNA, 23 bp, synthetic construct
  source 1..23: mol_type = other DNA
aggataaggccaaggtggaaatc

SEQ ID NO: 42 - DNA, 23 bp, synthetic construct
  source 1..23: mol_type = other DNA
gggatcagcgatatttcctgatg

SEQ ID NO: 43 - DNA, 23 bp, synthetic construct
  source 1..23: mol_type = other DNA
cagtaacgtgtacatgttgcaca

SEQ ID NO: 44 - DNA, 23 bp, synthetic construct
  source 1..23: mol_type = other DNA
ctgtaagactaacagcaaggatt

SEQ ID NO: 45 - DNA, 23 bp, synthetic construct
  source 1..23: mol_type = other DNA
aggttaactgtaatggcttcaca

SEQ ID NO: 46 - DNA, 23 bp, synthetic construct
  source 1..23: mol_type = other DNA
gcgatatttcctgatgttgcatt

SEQ ID NO: 47 - DNA, 23 bp, synthetic construct
  source 1..23: mol_type = other DNA
tggttgtttttggggaaaactgg

SEQ ID NO: 48 - DNA, 23 bp, synthetic construct
  source 1..23: mol_type = other DNA
gaccggttaagagattcttattt

SEQ ID NO: 49 - DNA, 23 bp, synthetic construct
  source 1..23: mol_type = other DNA
cacaattgaagatgaagaacttt

SEQ ID NO: 50 - DNA, 23 bp, synthetic construct
  source 1..23: mol_type = other DNA
ttcacaattgaagatgaagaact

SEQ ID NO: 51 - DNA, 23 bp, synthetic construct
  source 1..23: mol_type = other DNA
aactgtaatggcttcacaattga

SEQ ID NO: 52 - DNA, 23 bp, synthetic construct
  source 1..23: mol_type = other DNA
atgttgcattgatgaatcatagc

SEQ ID NO: 53 - DNA, 23 bp, synthetic construct
  source 1..23: mol_type = other DNA
aggacagtaacgtgtacatgttg

SEQ ID NO: 54 - DNA, 23 bp, synthetic construct
  source 1..23: mol_type = other DNA
aaggattctggccaaacagaaaa

SEQ ID NO: 55 - DNA, 23 bp, synthetic construct
  source 1..23: mol_type = other DNA
ccctgcaatatggacagaaaatc

SEQ ID NO: 56 - DNA, 23 bp, synthetic construct
  source 1..23: mol_type = other DNA
ttgaatcacatctggataagtta

SEQ ID NO: 57 - DNA, 23 bp, synthetic construct
  source 1..23: mol_type = other DNA
aagatgaagaactttctcatttg

SEQ ID NO: 58 - DNA, 23 bp, synthetic construct
  source 1..23: mol_type = other DNA
ggcttcacaattgaagatgaaga

SEQ ID NO: 59 - RNA, 21 nt, synthetic construct
  source 1..21: mol_type = other RNA
ccggttaagagattcttattt

SEQ ID NO: 60 - RNA, 21 nt, synthetic construct
  source 1..21: mol_type = other RNA
cggcaaagatcatccatattt

SEQ ID NO: 61 - RNA, 21 nt, synthetic construct
  source 1..21: mol_type = other RNA
cccaacggaagatagaaattt

SEQ ID NO: 62 - RNA, 21 nt, synthetic construct
  source 1..21: mol_type = other RNA
cctgcaatatggacagaaatt

SEQ ID NO: 63 - RNA, 21 nt, synthetic construct
  source 1..21: mol_type = other RNA
ggagactgtaagactaacatt

SEQ ID NO: 64 - RNA, 21 nt, synthetic construct
  source 1..21: mol_type = other RNA
gagctgtacaggaaatcaatt

SEQ ID NO: 65 - RNA, 21 nt, synthetic construct
  source 1..21: mol_type = other RNA
ccctgcaatatggacagaatt

SEQ ID NO: 66 - RNA, 21 nt, synthetic construct
  source 1..21: mol_type = other RNA
cggattccctgacaatgattt

SEQ ID NO: 67 - RNA, 21 nt, synthetic construct
  source 1..21: mol_type = other RNA
cagcgatatttcctgatgttt

SEQ ID NO: 68 - RNA, 21 nt, synthetic construct
  source 1..21: mol_type = other RNA
cacgcaacgtcattgaagatt

SEQ ID NO: 69 - RNA, 21 nt, synthetic construct
  source 1..21: mol_type = other RNA
caaagatcatccatatatttc

SEQ ID NO: 70 - RNA, 21 nt, synthetic construct
  source 1..21: mol_type = other RNA
ggtttttaccagctatattga

SEQ ID NO: 71 - RNA, 21 nt, synthetic construct
  source 1..21: mol_type = other RNA
ggttaagagattcttatttct

SEQ ID NO: 72 - RNA, 21 nt, synthetic construct
  source 1..21: mol_type = other RNA
gatcaaacaggaaattgaaag

SEQ ID NO: 73 - RNA, 21 nt, synthetic construct
  source 1..21: mol_type = other RNA
gataagttagacaatgagaag

SEQ ID NO: 74 - RNA, 20 nt, synthetic construct
  source 1..20: mol_type = other RNA
caatatggacagaaatcatt

SEQ ID NO: 75 - RNA, 21 nt, synthetic construct
  source 1..21: mol_type = other RNA
cggttaagagattcttatttc

SEQ ID NO: 76 - RNA, 21 nt, synthetic construct
  source 1..21: mol_type = other RNA
gattcttatttctttacctgt

SEQ ID NO: 77 - RNA, 21 nt, synthetic construct
  source 1..21: mol_type = other RNA
gcaaagatcatccatatattt

SEQ ID NO: 78 - RNA, 21 nt, synthetic construct
  source 1..21: mol_type = other RNA
gaaatgaccggttaagagatt

SEQ ID NO: 79 - RNA, 21 nt, synthetic construct
  source 1..21: mol_type = other RNA
gaaggagtttgaatcacattt

SEQ ID NO: 80 - RNA, 21 nt, synthetic construct
  source 1..21: mol_type = other RNA
ctcggagactgtaagactatt

SEQ ID NO: 81 - RNA, 21 nt, synthetic construct
  source 1..21: mol_type = other RNA
ctgtgtttgaggacagtaatt

SEQ ID NO: 82 - RNA, 21 nt, synthetic construct
  source 1..21: mol_type = other RNA
ccacctatattgatctccttt

SEQ ID NO: 83 - RNA, 21 nt, synthetic construct
  source 1..21: mol_type = other RNA
gcttcacaattgaagatgatt

SEQ ID NO: 84 - RNA, 21 nt, synthetic construct
  source 1..21: mol_type = other RNA
cacaattgaagatgaagaatt

SEQ ID NO: 85 - RNA, 21 nt, synthetic construct
  source 1..21: mol_type = other RNA
cttcacaattgaatatgaatt

SEQ ID NO: 86 - RNA, 21 nt, synthetic construct
  source 1..21: mol_type = other RNA
ctgtacccaacggaagatatt

SEQ ID NO: 87 - RNA, 21 nt, synthetic construct
  source 1..21: mol_type = other RNA
gtgtctgcttgtacatgcatt

SEQ ID NO: 88 - RNA, 21 nt, synthetic construct
  source 1..21: mol_type = other RNA
gcaatgtggagtgtcagaatt

SEQ ID NO: 89 - RNA, 21 nt, synthetic construct
  source 1..21: mol_type = other RNA
cgcaacgtcattgaagagttt

SEQ ID NO: 90 - RNA, 21 nt, synthetic construct
  source 1..21: mol_type = other RNA
ctgacaatgatagcctcgttt

SEQ ID NO: 91 - RNA, 21 nt, synthetic construct
  source 1..21: mol_type = other RNA
cccaatgtcattgtgaccttt

SEQ ID NO: 92 - RNA, 21 nt, synthetic construct
  source 1..21: mol_type = other RNA
gagaagatgagctctgtgttt

SEQ ID NO: 93 - RNA, 21 nt, synthetic construct
  source 1..21: mol_type = other RNA
catccatatatttctgagatt

SEQ ID NO: 94 - RNA, 21 nt, synthetic construct
  source 1..21: mol_type = other RNA
ccatatatttctgagatcatt

SEQ ID NO: 95 - RNA, 21 nt, synthetic construct
  source 1..21: mol_type = other RNA
gcattgatgaatcatagcttt

SEQ ID NO: 96 - RNA, 21 nt, synthetic construct
  source 1..21: mol_type = other RNA
gactgtaagactaacagcatt

SEQ ID NO: 97 - RNA, 21 nt, synthetic construct
  source 1..21: mol_type = other RNA
gattcagagtgacatagcttt

SEQ ID NO: 98 - RNA, 21 nt, synthetic construct
  source 1..21: mol_type = other RNA
gataaggccaaggtggaaatt

SEQ ID NO: 99 - RNA, 21 nt, synthetic construct
  source 1..21: mol_type = other RNA
gatcagcgatatttcctgatt

SEQ ID NO: 100 - RNA, 21 nt, synthetic construct
  source 1..21: mol_type = other RNA
gtaacgtgtacatgttgcatt

SEQ ID NO: 101 - RNA, 21 nt, synthetic construct
  source 1..21: mol_type = other RNA
gtaagactaacagcaaggatt

SEQ ID NO: 102 - RNA, 21 nt, synthetic construct
  source 1..21: mol_type = other RNA
gttaactgtaatggcttcatt

SEQ ID NO: 103 - RNA, 21 nt, synthetic construct
  source 1..21: mol_type = other RNA
gatatttcctgatgttgcatt

SEQ ID NO: 104 - RNA, 21 nt, synthetic construct
  source 1..21: mol_type = other RNA
gttgtttttggggaaaactgg

SEQ ID NO: 105 - RNA, 21 nt, synthetic construct
  source 1..21: mol_type = other RNA
ccggttaagagattcttattt

SEQ ID NO: 106 - RNA, 21 nt, synthetic construct
  source 1..21: mol_type = other RNA
caattgaagatgaagaacttt

SEQ ID NO: 107 - RNA, 21 nt, synthetic construct
  source 1..21: mol_type = other RNA
cacaattgaagatgaagaact

SEQ ID NO: 108 - RNA, 21 nt, synthetic construct
  source 1..21: mol_type = other RNA
ctgtaatggcttcacaattga

SEQ ID NO: 109 - RNA, 21 nt, synthetic construct
  source 1..21: mol_type = other RNA
gttgcattgatgaatcatagc

SEQ ID NO: 110 - RNA, 21 nt, synthetic construct
  source 1..21: mol_type = other RNA
gacagtaacgtgtacatgttg

SEQ ID NO: 111 - RNA, 21 nt, synthetic construct
  source 1..21: mol_type = other RNA
ggattctggccaaacagaaaa

SEQ ID NO: 112 - RNA, 21 nt, synthetic construct
  source 1..21: mol_type = other RNA
ctgcaatatggacagaaaatc

SEQ ID NO: 113 - RNA, 21 nt, synthetic construct
  source 1..21: mol_type = other RNA
gaatcacatctggataagtta

SEQ ID NO: 114 - RNA, 21 nt, synthetic construct
  source 1..21: mol_type = other RNA
gatgaagaactttctcatttg

SEQ ID NO: 115 - RNA, 21 nt, synthetic construct
  source 1..21: mol_type = other RNA
cttcacaattgaagatgaaga

SEQ ID NO: 116 - RNA, 21 nt, synthetic construct
  source 1..21: mol_type = other RNA
ataagaatctcttaaccggtt

SEQ ID NO: 117 - RNA, 21 nt, synthetic construct
  source 1..21: mol_type = other RNA
atatggatgatctttgccgtt

SEQ ID NO: 118 - RNA, 21 nt, synthetic construct
  source 1..21: mol_type = other RNA
atttctatcttccgttgggtt

SEQ ID NO: 119 - RNA, 21 nt, synthetic construct
  source 1..21: mol_type = other RNA
tttctgtccatattgcaggtt

SEQ ID NO: 120 - RNA, 21 nt, synthetic construct
  source 1..21: mol_type = other RNA
tgttagtcttacagtctcctt

SEQ ID NO: 121 - RNA, 21 nt, synthetic construct
  source 1..21: mol_type = other RNA
ttgatttcctgtacagctctt

SEQ ID NO: 122 - RNA, 21 nt, synthetic construct
  source 1..21: mol_type = other RNA
ttctgtccatattgcagggtt

SEQ ID NO: 123 - RNA, 21 nt, synthetic construct
  source 1..21: mol_type = other RNA
atcattgtcagggaatccgtt

SEQ ID NO: 124 - RNA, 21 nt, synthetic construct
  source 1..21: mol_type = other RNA
acatcaggaaatatcgctgtt

SEQ ID NO: 125 - RNA, 21 nt, synthetic construct
  source 1..21: mol_type = other RNA
tcttcaatgacgttgcgtgtt

SEQ ID NO: 126 - RNA, 21 nt, synthetic construct
  source 1..21: mol_type = other RNA
aatatatggatgatctttgcc

SEQ ID NO: 127 - RNA, 21 nt, synthetic construct
  source 1..21: mol_type = other RNA
aatatagctggtaaaaacctc

SEQ ID NO: 128 - RNA, 21 nt, synthetic construct
  source 1..21: mol_type = other RNA
aaataagaatctcttaaccgg

SEQ ID NO: 129 - RNA, 21 nt, synthetic construct
  source 1..21: mol_type = other RNA
ttcaatttcctgtttgatctc

SEQ ID NO: 130 - RNA, 21 nt, synthetic construct
  source 1..21: mol_type = other RNA
tctcattgtctaacttatcca

SEQ ID NO: 131 - RNA, 21 nt, synthetic construct
  source 1..21: mol_type = other RNA
tgattttctgtccatattgca

SEQ ID NO: 132 - RNA, 21 nt, synthetic construct
  source 1..21: mol_type = other RNA
aataagaatctcttaaccggt

SEQ ID NO: 133 - RNA, 21 nt, synthetic construct
  source 1..21: mol_type = other RNA
aggtaaagaaataagaatctc

SEQ ID NO: 134 - RNA, 21 nt, synthetic construct
  source 1..21: mol_type = other RNA
atatatggatgatctttgccg

SEQ ID NO: 135 - RNA, 21 nt, synthetic construct
  source 1..21: mol_type = other RNA
tctcttaaccggtcatttcta

SEQ ID NO: 136 - RNA, 21 nt, synthetic construct
  source 1..21: mol_type = other RNA
atgtgattcaaactccttctt

SEQ ID NO: 137 - RNA, 21 nt, synthetic construct
  source 1..21: mol_type = other RNA
tagtcttacagtctccgagtt

SEQ ID NO: 138 - RNA, 21 nt, synthetic construct
  source 1..21: mol_type = other RNA
ttactgtcctcaaacacagtt

SEQ ID NO: 139 - RNA, 21 nt, synthetic construct
  source 1..21: mol_type = other RNA
aggagatcaatatagctggtt

SEQ ID NO: 140 - RNA, 21 nt, synthetic construct
  source 1..21: mol_type = other RNA
tcatcttcaattgtgaagctt

SEQ ID NO: 141 - RNA, 21 nt, synthetic construct
  source 1..21: mol_type = other RNA
ttcttcatcttcaattgtgtt

SEQ ID NO: 142 - RNA, 21 nt, synthetic construct
  source 1..21: mol_type = other RNA
ttcatcttcaattgtgaagtt

SEQ ID NO: 143 - RNA, 21 nt, synthetic construct
  source 1..21: mol_type = other RNA
tatcttccgttgggtacagtt

SEQ ID NO: 144 - RNA, 21 nt, synthetic construct
  source 1..21: mol_type = other RNA
tgcatgtacaagcagacactt

SEQ ID NO: 145 - RNA, 21 nt, synthetic construct
  source 1..21: mol_type = other RNA
ttctgacactccacattgctt

SEQ ID NO: 146 - RNA, 21 nt, synthetic construct
  source 1..21: mol_type = other RNA
actcttcaatgacgttgcgtt

SEQ ID NO: 147 - RNA, 21 nt, synthetic construct
  source 1..21: mol_type = other RNA
acgaggctatcattgtcagtt

SEQ ID NO: 148 - RNA, 21 nt, synthetic construct
  source 1..21: mol_type = other RNA
aggtcacaatgacattgggtt

SEQ ID NO: 149 - RNA, 21 nt, synthetic construct
  source 1..21: mol_type = other RNA
acacagagctcatcttctctt

SEQ ID NO: 150 - RNA, 21 nt, synthetic construct
  source 1..21: mol_type = other RNA
tctcagaaatatatggatgtt

SEQ ID NO: 151 - RNA, 21 nt, synthetic construct
  source 1..21: mol_type = other RNA
tgatctcagaaatatatggtt

SEQ ID NO: 152 - RNA, 21 nt, synthetic construct
  source 1..21: mol_type = other RNA
agctatgattcatcaatgctt

SEQ ID NO: 153 - RNA, 21 nt, synthetic construct
  source 1..21: mol_type = other RNA
tgctgttagtcttacagtctt

SEQ ID NO: 154 - RNA, 21 nt, synthetic construct
  source 1..21: mol_type = other RNA
agctatgtcactctgaatctt

SEQ ID NO: 155 - RNA, 21 nt, synthetic construct
  source 1..21: mol_type = other RNA
tttccaccttggccttatctt

SEQ ID NO: 156 - RNA, 21 nt, synthetic construct
  source 1..21: mol_type = other RNA
tcaggaaatatcgctgatctt

SEQ ID NO: 157 - RNA, 21 nt, synthetic construct
  source 1..21: mol_type = other RNA
tgcaacatgtacacgttactt

SEQ ID NO: 158 - RNA, 21 nt, synthetic construct
  source 1..21: mol_type = other RNA
tccttgctgttagtcttactt

SEQ ID NO: 159 - RNA, 21 nt, synthetic construct
  source 1..21: mol_type = other RNA
tgaagccattacagttaactt

SEQ ID NO: 160 - RNA, 21 nt, synthetic construct
  source 1..21: mol_type = other RNA
tgcaacatcaggaaatatctt

SEQ ID NO: 161 - RNA, 21 nt, synthetic construct
  source 1..21: mol_type = other RNA
agttttccccaaaaacaacca

SEQ ID NO: 162 - RNA, 21 nt, synthetic construct
  source 1..21: mol_type = other RNA
ataagaatctcttaaccggtc

SEQ ID NO: 163 - RNA, 21 nt, synthetic construct
  source 1..21: mol_type = other RNA
agttcttcatcttcaattgtg

SEQ ID NO: 164 - RNA, 21 nt, synthetic construct
  source 1..21: mol_type = other RNA
ttcttcatcttcaattgtgaa

SEQ ID NO: 165 - RNA, 21 nt, synthetic construct
  source 1..21: mol_type = other RNA
aattgtgaagccattacagtt

SEQ ID NO: 166 - RNA, 21 nt, synthetic construct
  source 1..21: mol_type = other RNA
tatgattcatcaatgcaacat

SEQ ID NO: 167 - RNA, 21 nt, synthetic construct
  source 1..21: mol_type = other RNA
acatgtacacgttactgtcct

SEQ ID NO: 168 - RNA, 21 nt, synthetic construct
  source 1..21: mol_type = other RNA
ttctgtttggccagaatcctt

SEQ ID NO: 169 - RNA, 21 nt, synthetic construct
  source 1..21: mol_type = other RNA
ttttctgtccatattgcaggg

SEQ ID NO: 170 - RNA, 21 nt, synthetic construct
  source 1..21: mol_type = other RNA
acttatccagatgtgattcaa

SEQ ID NO: 171 - RNA, 21 nt, synthetic construct
  source 1..21: mol_type = other RNA
aatgagaaagttcttcatctt

SEQ ID NO: 172 - RNA, 21 nt, synthetic construct
  source 1..21: mol_type = other RNA
ttcatcttcaattgtgaagcc

SEQ ID NO: 173 - DNA, 1299 bp, Homo sapiens
  source 1..1299: mol_type = genomic DNA
agggccgagggcctcggcggcctggagcgcttctgcagcccgggcaaaggccgggggctgcgggctctgcagcccttccaggtgggggacttgctgttctcctgcccggcctatgcctacgtgctcacggtcaacgagcggggcaaccactgcgagtactgcttcaccaggaaagaaggattgtccaaatgtggaagatgcaagcaggcattttactgcaatgtggagtgtcagaaagaagattggcccatgcacaagctggaatgttctcccatggttgtttttggggaaaactggaatccctcggagactgtaagactaacagcaaggattctggccaaacagaaaatccacccagagagaacaccttcggaaaaattgttagctgtgaaggagtttgaatcacatctggataagttagacaatgagaagaaggatttgattcagagtgacatagctgctctccatcacttttactccaagcatctcggattccctgacaatgatagcctcgtagtactctttgcacaggttaactgtaatggcttcacaattgaagatgaagaactttctcatttgggatcagcgatatttcctgatgttgcattgatgaatcatagctgttgccccaatgtcattgtgacctacaaagggaccctggcagaagtcagagctgtacaggaaatcaagccgggagaggaggtttttaccagctatattgatctcctgtacccaacggaagatagaaatgaccggttaagagattcttatttctttacctgtgagtgccaggagtgtaccaccaaggacaaggataaggccaaggtggaaatccggaagctcagcgatcccccaaaggcagaagccatccgagacatggtcagatatgcacgcaacgtcattgaagagttccggagggccaagcactataaatcccctagtgagctgctggagatctgcgagctcagccaggagaagatgagctctgtgtttgaggacagtaacgtgtacatgttgcacatgatgtaccaggccatgggtgtctgcttgtacatgcaggactgggaaggagccctgcaatatggacagaaaatcattaagccctacagtaagcactatcctttgtactccctcaacgtggcctccatgtggttgaagctagggagactctacatgggcctggaacacaaagccgcaggggagaaagccctgaagaaggccattgcaatcatggaagtagctcacggcaaagatcatccatatatttctgagatcaaacaggaaattgaaagccactga

(19) United States
(12) Patent Application Publication
(10) Pub. No.: US 2025/0257361 A1
(43) Pub. Date: Aug. 14, 2025

(54) COMPOSITIONS AND METHODS FOR TREATING LIVER DISEASES WITH SIRNAS TARGETING SMYD2

(71) Applicant: The Board of Regents of The University of Texas System, Dallas, TX (US)

(72) Inventors: Hao Zhu, Dallas, TX (US); Zixi Wang, Dallas, TX (US); Lin Li, Flower Mound, TX (US)

(21) Appl. No.: 18/866,952

(22) PCT Filed: Oct. 6, 2023
(86) PCT No.: PCT/US2023/076222

§ 371 Date: Nov. 18, 2024

Related U.S. Application Data
(60) Provisional application No. 63/382,920, filed on Nov. 9, 2022.

(51) Int. Cl. C12N 15/113

(52) U.S. Cl. CPC C12N 15/1138; C12Y 201/01; C12N 2310/14; C12N 2310/351

(57) ABSTRACT

Disclosed herein are compositions comprising siRNAs capable of downregulating SET and MYND (Myeloid-Nervy-DEAF1) domain-containing protein 2 (SMYD2) gene expression or a variant thereof. Also disclosed herein are methods of using SMYD2 inhibitors in the treatment of a liver disease or injury, such as fatty liver disease (FLD), non-alcoholic fatty liver disease (NAFLD) or non-alcoholic steatohepatitis (NASH).

### CROSS-REFERENCE TO RELATED APPLICATION

This application claims priority to U.S. Provisional Patent Application Ser. No. 63/382,920, filed Nov. 9, 2022, and titled “COMPOSITIONS AND METHODS FOR TREATING LIVER DISEASES WITH SIRNAS TARGETING SMYD2,” which is incorporated by reference herein in its entirety.

### INCORPORATION BY REFERENCE OF SEQUENCE LISTING

This application contains a Sequence Listing that has been submitted in WIPO ST.26.xml format via Patent Center and is hereby incorporated by reference in its entirety. The .xml copy is named “106546-774250-4136_Sequence_Listing.xml” and is 153 KB in size.

### BACKGROUND

### 1. Field

The present inventive concept is directed to compositions and methods of use thereof for liver disease treatment, specifically to metabolic liver diseases, including but not limited to non-alcoholic fatty liver disease (NAFLD) and nonalcoholic steatohepatitis (NASH).

### 2. Discussion of Related Art

NAFLD is a spectrum of chronic liver disorders, which encompass, among others nonalcoholic fatty liver (NAFL) and nonalcoholic steatohepatitis (NASH). NASH, a common cause of chronic liver disease, is defined as having at least 5% hepatic steatosis and inflammation with or without fibrosis. Over time and without treatment, NASH may progress to cirrhosis and even hepatocellular carcinoma (HCC). Moreover, NAFLD/NASH is strongly associated with obesity and type II diabetes which together affect over 50% of the US population, leading to a heavy economic burden. Unfortunately, therapeutic options for NASH remain limited, with only slight benefits observed from vitamin E or obeticholic acid treatment. While NASH is the leading cause of chronic liver disease and cirrhosis, there are currently no clinically approved therapies. As such, new targets, therapeutics, and combinations thereof are needed to accelerate clinical progress in the treatment of liver diseases.

### SUMMARY

In one aspect, the present disclosure discloses a small interfering RNA (siRNA) molecule that specifically downregulates gene expression of SMYD2 (SET and Myeloid-Nervy-DEAF-1 domain-containing protein 2) or a variant thereof. The siRNA molecule may comprise a nucleotide sequence that is 2 to 30 nucleotides in length and is at least 80% homologous to at least 2 to 30 contiguous nucleotides of a human SMYD2 cDNA sequence. In another aspect, the present disclosure discloses a siRNA molecule targets the open reading frame or the 5′ or 3′ UTRs of the human SMYD2 gene. In yet another aspect, the siRNA molecule may comprise at least one sense sequence, at least one antisense sequence, or both a sense and an antisense sequence.

In one aspect, the present disclosure discloses a siRNA molecule downregulates human SMYD2 gene expression, including but not limited to, reduced expression for at least about 30%, at least about 40%, at least about 50%, at least about 60%, at least about 70%, at least about 80%, or at least about 90% of human SMYD2 gene expression. In another aspect, the siRNA molecule specifically downregulates gene expression of at least one variant of the human SMYD2. In yet another aspect, the siRNA molecule may downregulate the human SMYD2 gene associated with a liver disease.

In one aspect, the siRNA molecule comprises a part of human SMYD2 cDNA sequence is SEQ ID NO: 1. In one aspect, the siRNA molecule comprises at least one nucleotide sequence that is 2 to 30 nucleotides in length and is at least 80% homologous to at least one of the cDNA sequences of SEQ ID Nos.: 2-58. In another aspect, the siRNA molecule may comprise at least one nucleotide sequence that is 2 to 30 nucleotides in length and is at least 80% homologous to at least one of the cDNA sequences of SEQ ID Nos.: 9, 11, or 12. In yet another aspect, the siRNA molecule may comprise a nucleotide sequence of SEQ ID Nos: 59-172, a nucleotide sequence having at least 80% identity to any one of SEQ ID NOs: 59-172, or any combination thereof. In yet another aspect, the siRNA molecule may comprise a nucleotide sequence having at least 90% identity of any one of Seq ID Nos.: 66, 68, 69, 123, 125, 126, or any combination thereof. In one aspect, the siRNA molecule may comprise a nucleotide sequence of any one of SEQ ID NOs.: 59-115. In another aspect, the siRNA molecule may comprise at least one antisense sequence of a nucleotide sequence of any one of SEQ ID NOs.: 116-172.

In one aspect, the present disclosure discloses a siRNA molecule is conjugated to least one targeting ligand. In one aspect, the siRNA molecule may comprise at least one liver targeting ligand. In another aspect, the liver targeting ligand comprises at least one N-Acetylgalactosamine (GalNAc) conjugate. In one aspect, the siRNA molecule may be conjugated to about one to about three GalNAc conjugates. In another aspect, the siRNA molecule comprises at least one chemical modification. In one aspect, the siRNA molecule may comprise a modification with at least one ribosugar moiety of its nucleotide sequence. In yet another aspect, the siRNA molecule may include least one ribosugar moiety modified with 2 2′-O-methyl (2′OMe), 2′-deoxy-2′-fluoro (2′F), 2′-deoxy, 5-C-methyl, 2′-O-(2-methoxyethyl) (MOE), 4′-thio, 2′-amino, 2′-C-allyl, or any combination thereof. In yet another aspect, the siRNA molecule comprises less than about 10% to about 70% of modified ribosugar moieties in the total nucleotide sequence.

In one aspect, the present disclosure discloses a composition comprises the siRNA molecules, and at least one excipient. In another aspect, the composition is a pharmaceutical composition and the at least one excipient is at least one pharmaceutically acceptable carrier. In yet another aspect, the pharmaceutical composition may comprise a nanoparticle. In yet another aspect, the pharmaceutical composition may comprise a lipid. In another aspect, the method of administering comprises parenteral administration.

In one aspect, the present disclosure discloses a method of treating a subject in need thereof. The method comprises administrating a therapeutically effective amount of the siRNA a composition thereof, or a pharmaceutical composition thereof to the subject. In one aspect, the method comprises a human subject having or suspected of having a liver disease. In another aspect, the liver disease comprises fatty liver disease (FLD), alcohol-related liver disease (ARLD), non-alcoholic fatty liver disease (NAFLD), non-alcoholic steatohepatitis (NASH), end stage liver disease (cirrhosis) from any etiology, liver cancer, or any combination thereof.

In one aspect, the present disclosure discloses a kit comprising a container holding the siRNA, the composition thereof, or the pharmaceutical composition thereof. The kit may also comprise a pharmaceutical administrative means and an instruction.

In one aspect, the present disclosure discloses a method of administration of a therapeutically effective amount of the siRNA, the composition thereof, or the pharmaceutical composition thereof to increase life expectancy of the subject compared to an untreated subject with identical disease condition and predicted outcome.

In one aspect, the present disclosure discloses a method of administration of a therapeutically effective amount of the siRNA, the composition thereof, or the pharmaceutical composition thereof to improve liver function of the subject compared to an untreated subject with identical disease condition and predicted outcome.

In one aspect, the present disclosure discloses a method of administration of a therapeutically effective amount of the siRNA, the composition thereof, or the pharmaceutical composition thereof to attenuate liver fibrosis in the subject compared to an untreated subject with identical disease condition and predicted outcome.

Other technical features may be readily apparent to one skilled in the art from the following figures, descriptions, and claims.

### BRIEF DESCRIPTION OF THE DRAWINGS

The application file contains at least one drawing executed in color. Copies of this patent application publication with color drawing(s) will be provided by the Office upon request and payment of the necessary fee.

The following drawings form part of the present specification and are included to further demonstrate certain aspects of the present disclosure, which can be better understood by reference to the drawing in combination with the detailed description of specific embodiments presented herein. Embodiments of the present inventive concept are illustrated by way of example in which like reference numerals indicate similar elements and in which:

FIG. 1 depicts lineage tracing of mosaic mutant hepatocytes demonstrated that mutations that suppress lipogenesis become enriched in fatty livers. Schema for pooled tracing of mosaic mutant hepatocytes under different dietary conditions. MOSAICS AAVs carrying sgRNA libraries were injected into Cas9 expressing mice. Ten days after gene perturbation, Cas9 expression was turned off by dox withdrawal, and chow or WD was given to mice for another 6 months. Genomic DNA was extracted from harvested livers. sgRNA sequences were amplified and quantified. sgRNAs that were specifically enriched in fatty but not normal livers were further investigated. This approach aimed to exclude sgRNAs that target proliferation suppressing genes which would manifest as enrichment in both normal and fatty livers.

FIG. 2 depicts the workflow for identifying differentially activated (for transcription factors) or expressed (for epigenetic proteins) proteins in NAFLD and HCV cirrhosis patients.

FIGS. 3A-3M depict Somatic mosaic screening of transcription and epigenetic factors identifies putative therapeutic targets for NASH. FIG. 3A. Results for somatic mosaic screening of transcription factors. MOSAICS vectors carrying transcription factor targeting sgRNA libraries were injected into Cas9 expressing mice. Chow or WD was fed to mice for 6 months. The genes corresponding to enriched sgRNAs in WD fed but not chow fed livers were drawn as colored circles with sizes correlating to −log2(p). Control sgRNAs were drawn as filled black circles. FIG. 3B. Results for somatic mosaic screening of epigenetic proteins. The screening method and color scheme are the same as in FIG. 3A. FIG. 3C. List of genes corresponding to enriched sgRNAs (p<0.05) in both MOSAICS screens. FIG. 3D Schematic for examining the KO phenotypes of the top genes under WD conditions. A MOSAICS AAV carrying an individual sgRNA was injected into Cas9 mice such that each mouse had one gene deleted in the liver. Dox was withdrawn 10 days after AAV injection and WD was given for 3 months before sacrifice. FIG. 3E-3F. Body weight and liver/body ratios of control (sgGFP and sgLacZ) and liver-specific KO mice fed with 3 months of WD. Gray dots represent control mice, blue dots represent liver-specific KO mice for known NASH genes, red dots represent liver-specific transcription factor KO mice, and green dots represent liver-specific epigenetic factor KO mice. Darker dots represent mice that have the most significant differences in liver/body weight ratios. Each dot represents one mouse, and the n is denoted at the bottom of each plot. FIG. 3G. Liver function analysis using plasma ALT. The color scheme is the same as in FIG. 3E. FIGS. 3H-3I. Liver triglyceride and cholesterol analysis. FIG. 3J. Liver weight of control (sgGFP and sgLacZ) and liver-specific KO mice fed with 3 months of WD. The color scheme is the same as in FIG. 3E. Each dot represents one mouse. The n is denoted at the bottom of each plot. FIG. 3K-3M. Smyd2 liver KO mice have reduced ALT, plasma ALT, plasma triglyceride, and plasma cholesterol, all indications of reduced fatty liver disease.

FIG. 4 depicts representative H&E staining of liver sections from the control mice and the Smyd2 liver KO mice and fed with 3 months of western diets and sugar water. H&E staining of liver sections from two types of control KO mice (First two images) and Smyd2 liver specific KO mice (Third image).

FIGS. 5A-5F depict Transcriptional analysis of Irs1, Srebf1, Bcl6, Tbx3, and Smyd2 KO livers after WD. FIG. 5A. The number of genes with altered expression in the RNA-seq data when comparing control (sgGFP and sgLacZ) and KO livers after 3 months of WD. Darker and lighter colored bars represent the number of differentially expressed genes with a fold change of >=2 and >=1.5, respectively. Genes with statistically significant fold change differences of less than 1.5 are not included here. FIG. 5B. Heatmaps showing the fold changes of differentially expressed genes in fatty acid, triglyceride, and cholesterol synthesis pathways. The average expression level of control samples (four sgGFP and two sgLacZ) were normalized to 1 for each gene. FIG. 5C. Heatmaps showing the fold changes of differentially expressed genes in β-oxidation and TCA cycle pathways. The normalization method is the same as shown in FIGS. 5B. FIG. 5D. Hallmark pathway enrichment analysis of RNA-seq data from A. FIG. 5E. Heatmaps showing the fold changes of differentially expressed genes in fatty acid, triglyceride, and cholesterol synthesis pathways. The average expression level of control samples (four sgGFP and two sgLacZ) were normalized to 1 for each gene. FIG. 5F Heatmaps showing the fold changes of differentially expressed genes in β-oxidation and TCA cycle pathways. The normalization method is the same as shown in FIG. 5E.

FIGS. 6A-6K depict that AZ505, a selective SMYD2 inhibitor, ameliorates fatty liver disease. FIG. 6A. Experimental design for pre-clinical testing of AZ505 in the WD NASH model. WD was given at 8 weeks of age for a total of 2 months. AZ505 treatment started one day after WD was initiated. Vehicle (2.5% DMSO in saline) or AZ505 (10 mg/kg in saline containing 2.5% DMSO) was given to mice intraperitoneally 3 times per week. FIG. 6B. Body weight of vehicle or AZ505 treated mice before and after 2 months of WD (n=15 and 14 mice). FIGS. 6C-6D. Liver weight and liver/body weight ratios of vehicle or AZ505 treated mice after 2 months of WD (n=15 and 14). FIG. 6E. Representative liver pictures and H&E staining of liver sections for vehicle or AZ505 treated mice after 2 months of WD. FIGS. 6F-6G. Liver function analysis using plasma ALT and AST (n=15 and 14). FIGS. 6H-6I. Liver triglyceride and cholesterol analysis (n=15 and 14). FIGS. 6J-6K. Plasma triglyceride and cholesterol analysis (n=15 and 14).

FIG. 7 depicts human Smyd2 luciferase reporter plasmid construct used for siRNA testing.

FIG. 8 depicts efficacy of 20 siRNAs designed and tested in SMYD2 luciferase reporter assay in Cos7 cells.

The drawing figures do not limit the present inventive concept to the specific embodiments disclosed and described herein. The drawings are not necessarily to scale, emphasis instead being placed on clearly illustrating principles of certain embodiments of the present inventive concept.

### DETAILED DESCRIPTION

The following detailed description references the accompanying drawings that illustrate various embodiments of the present inventive concept. The drawings and description are intended to describe aspects and embodiments of the present inventive concept in sufficient detail to enable those skilled in the art to practice the present inventive concept. Other components can be utilized and changes can be made without departing from the scope of the present inventive concept. The following description is, therefore, not to be taken in a limiting sense. The scope of the present inventive concept is defined only by the appended claims, along with the full scope of equivalents to which such claims are entitled.

With increasing over-nutrition and obesity, non-alcoholic fatty liver disease (NAFLD) is rapidly becoming the leading cause of liver disease in the world. NAFLD is usually conceptualized at the organismal and tissue levels; however, little thought has been given to genetic heterogeneity within clones of the liver. Somatic mutations are common in most healthy individuals, and there is accumulating evidence that mutation burden increases with age and chronic tissue damage. The present disclosure is based, in part, on the novel finding that positive selection of somatic mutations in NASH patient livers can increase cell fitness and competitiveness through decreased lipid accumulation in NASH. Based on this novel concept, vivo genetic screening of somatically mutated transcriptional regulators dysregulated in human liver disease was performed. This genetic screen identified genes that when mutated, promoted clonal fitness through the reversal of lipotoxicity, including SMYD2 (SET and MYND (Myeloid-Nervy-DEAF1) domain-containing protein 2 (SMYD2)). Accordingly, provided herein are compositions and methods of use thereof for liver disease treatment, specifically to metabolic liver diseases, including but not limited to, non-alcoholic fatty liver disease (NAFLD) and nonalcoholic steatohepatitis (NASH).

### I. Terminology

The phraseology and terminology employed herein are for the purpose of description and should not be regarded as limiting. For example, the use of a singular term, such as, “a” is not intended as limiting of the number of items. Also, the use of relational terms such as, but not limited to, “top,” “bottom,” “left,” “right,” “upper,” “lower,” “down,” “up,” and “side,” are used in the description for clarity in specific reference to the figures and are not intended to limit the scope of the present inventive concept or the appended claims.

Further, as the present inventive concept is susceptible to embodiments of many different forms, it is intended that the present disclosure be considered as an example of the principles of the present inventive concept and not intended to limit the present inventive concept to the specific embodiments shown and described. Any one of the features of the present inventive concept may be used separately or in combination with any other feature. References to the terms “aspect”, “aspects”, “embodiment,” “embodiments,” and/or the like in the description mean that the feature and/or features being referred to are included in, at least, one aspect of the description. Separate references to the terms “aspect”, “aspects”, “embodiment,” “embodiments,” and/or the like in the description do not necessarily refer to the same embodiment and are also not mutually exclusive unless so stated and/or except as will be readily apparent to those skilled in the art from the description. For example, a feature, structure, process, step, action, or the like described in one embodiment may also be included in other embodiments but is not necessarily included. Thus, the present inventive concept may include a variety of combinations and/or integrations of the embodiments described herein. Additionally, all aspects of the present disclosure, as described herein, are not essential for its practice. Likewise, other systems, methods, features, and advantages of the present inventive concept will be, or become, apparent to one with skill in the art upon examination of the figures and the description. It is intended that all such additional systems, methods, features, and advantages be included within this description, be within the scope of the present inventive concept, and be encompassed by the claims.

As used herein, the term “about,” can mean relative to the recited value, e.g., amount, dose, temperature, time, percentage, etc., ±10%, ±9%, ±8%, ±7%, ±6%, ±5%, ±4%, ±3%, ±2%, or ±1%.

The terms “comprising,” “including,” “encompassing” and “having” are used interchangeably in this disclosure. The terms “comprising,” “including,” “encompassing” and “having” mean to include, but not necessarily be limited to the things so described.

The terms “or” and “and/or,” as used herein, are to be interpreted as inclusive or meaning any one or any combination. Therefore, “A, B or C” or “A, B and/or C” mean any of the following: “A,” “B” or “C”; “A and B”; “A and C”; “B and C”; “A, B and C.” An exception to this definition will occur only when a combination of elements, functions, steps or acts are in some way inherently mutually exclusive.

“Biomarker” as used herein refers to any biological molecules (e.g., nucleic acids, genes, peptides, proteins, lipids, hormones, metabolites, and the like) that, singularly or collectively, reflect the current or predict future state of a biological system. Thus, as used herein, the presence or concentration of one or more biomarkers can be detected and correlated with a known condition, such as a disease state. In some aspects, detecting the presence and/or concentration of one or more biomarkers herein may be an indication of a liver disease risk in a subject. In some other aspects, detecting the presence and/or concentration of one or more biomarkers herein may be used in treating and/or preventing a chronic liver disease in a subject.

As used herein, the terms “treat”, “treating”, “treatment” and the like, unless otherwise indicated, can refer to reversing, alleviating, inhibiting the process of, or preventing the disease, disorder or condition to which such term applies, or one or more symptoms of such disease, disorder or condition and includes the administration of any of the compositions, pharmaceutical compositions, or dosage forms described herein, to prevent the onset of the symptoms or the complications, or alleviating the symptoms or the complications, or eliminating the condition, or disorder.

The term “biomolecule” as used herein refers to, but is not limited to, proteins, enzymes, antibodies, DNA, siRNA, and small molecules. “Small molecules” as used herein can refer to chemicals, compounds, drugs, and the like.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. A polypeptide includes a natural peptide, a recombinant peptide, or a combination thereof.

It should also be understood that, unless clearly indicated to the contrary, in any methods claimed herein that include more than one step or act, the order of the steps or acts of the method is not necessarily limited to the order in which the steps or acts of the method are recited.

### II. Compositions

### (a) SMYD2

The SET (Suppressor of variegation, Enhancer of Zeste, Trithorax) and MYND (Myeloid-Nervy-DEAF1) domain-containing proteins (SMYD) have been found to methylate a variety of histone and non-histone targets which contribute to their various roles in cell regulation including chromatin remodeling, transcription, signal transduction, and cell cycle control. During early development, SMYD proteins are believed to act as an epigenetic regulator for myogenesis and cardio-myocyte differentiation as they are abundantly expressed in cardiac and skeletal muscle. The present disclosure provides for novel compositions for modulating the SMYD2 (SET and MYND domain containing 2) gene expression and methods of preventing, attenuating, and/or treating liver diseases.

In certain embodiments, compositions for use in the methods disclosed herein can modulate SMYD2. As used herein, compositions “modulating” SMYD2 can include any biomolecule(s) capable of decreasing SMYD2 gene expression, decreasing SMYD2 protein expression, decreasing SMYD2 activity, or a combination thereof. In some aspects, biomolecule(s) herein capable of modulating SMYD2 can be an inhibitor of SMYD2. As used herein, an inhibitor of SMYD2 can inhibit SMYD2 direct activity, inhibit SMYD2 indirect activity, decrease expression of the SMYD2 gene, decrease expression of the SMYD2 protein, or a combination thereof.

In certain embodiments, compositions for use in the methods disclosed herein can include a nucleic acid molecule. The term “nucleic acid molecule” as used herein refers to a molecule having nucleotides. The nucleic acid can be single, double, or multiple stranded and may comprise modified or unmodified nucleotides or non-nucleotides or various mixtures and combinations thereof. In some embodiments, a nucleic acid molecule for use herein can be a double-stranded RNA. In some examples, a double stranded RNA suitable for use herein can be small temporal RNA, small nuclear RNA, small nucleolar RNA, short hairpin RNA, microRNA, or the like. In certain embodiments, a double stranded RNA suitable for use herein can be a small interfering RNA (siRNA).

The term “siRNA” as used herein refers to small inhibitory RNA duplexes that induce the RNA interference (RNAi) pathway. A siRNA molecule disclosed herein may be capable of silencing, reducing, and/or inhibiting expression of a target gene (e.g., SMYD2). These molecules can vary in length (generally about 5-50 base pairs) and contain varying degrees of complementarity to their target mRNA in the antisense strand. Some, but not all, siRNA have unpaired overhanging bases on the 5′ or 3′ end of the sense strand and/or the antisense strand. The term “siRNA” as used herein can include duplexes of two separate strands, as well as single strands that can form hairpin structures comprising a duplex region.

siRNA molecules disclosed herein may be any interfering RNA with a duplex length of about 2-60, about 5-50, or about 10-40 nucleotides in length, more typically about 2-30, about 5-25, or about 10-25 nucleotides in length. In some embodiments, siRNA molecules disclosed herein may have a nucleotide sequence that is about 2 to about 30 nucleotides in length, about 5 to about 25 nucleotides in length, or about 10 to about 20 nucleotides in length. In some embodiments, siRNA molecules disclosed herein may have a nucleotide sequence that is about 10 nucleotides in length, about 11 nucleotides in length, about 12 nucleotides in length, about 13 nucleotides in length, about 14 nucleotides in length, about 15 nucleotides in length, about 14 nucleotides in length, about 15 nucleotides in length, about 16 nucleotides in length, about 17 nucleotides in length, about 18 nucleotides in length, about 19 nucleotides in length, about 20 nucleotides in length, about 21 nucleotides in length, about 22 nucleotides in length, about 23 nucleotides in length, about 24 nucleotides in length, or about 25 nucleotides in length. Each complementary sequence of a double-stranded siRNA disclosed herein may be about 2-60, about 5-50, about 10-40, about 2-30, about 5-25, or about 10-25 nucleotides in length, but other noncomplementary sequences may be present. For example, siRNA duplexes may comprise 3′ overhangs of about 1 to about 4 or more nucleotides and/or 5′ phosphate termini comprising about 1 to about 4 or more nucleotides.

In certain embodiments, siRNA molecules disclosed herein may have about 2-60, about 2-50, about 2-40, or about 2-30 contiguous nucleotides of homology with a target (e.g., SMYD2) nucleotide sequence. In some aspects, a target nucleotide sequence herein may be a human SMYD2 nucleotide sequence or a variant thereof. In some other aspects, a target nucleotide sequence herein may be human SMYD2, RefSeq: NM_020197.3, or a variant thereof. In still some other aspects, a target nucleotide sequence herein may be SEQ ID NO: 1 as follows, or a variant thereof:

Human SMYD2 cDNA (SEQ ID NO: 1):
ATGAGGGCCGAGGGCCTCGGCGGCCTGGAGCGCTTCTGCAGCCCGG
GCAAAGGCCGGGGGCTGCGGGCTCTGCAGCCCTTCCAGGTGGGGG
ACTTGCTGTTCTCCTGCCCGGCCTATGCCTACGTGCTCACGGTCA
ACGAGCGGGGCAACCACTGCGAGTACTGCTTCACCAGGAAAGAAG
GATTGTCCAAATGTGGAAGATGCAAGCAGGCATTTTACTGCAATG
TGGAGTGTCAGAAAGAAGATTGGCCCATGCACAAGCTGGAATGTT
CTCCCATGGTTGTTTTTGGGGAAAACTGGAATCCCTCGGAGACTG
TAAGACTAACAGCAAGGATTCTGGCCAAACAGAAAATCCACCCAG
AGAGAACACCTTCGGAAAAATTGTTAGCTGTGAAGGAGTTTGAAT
CACATCTGGATAAGTTAGACAATGAGAAGAAGGATTTGATTCAGA
GTGACATAGCTGCTCTCCATCACTTTTACTCCAAGCATCTCGGAT
TCCCTGACAATGATAGCCTCGTAGTACTCTTTGCACAGGTTAACT
GTAATGGCTTCACAATTGAAGATGAAGAACTTTCTCATTTGGGAT
CAGCGATATTTCCTGATGTTGCATTGATGAATCATAGCTGTTGCC
CCAATGTCATTGTGACCTACAAAGGGACCCTGGCAGAAGTCAGAG
CTGTACAGGAAATCAAGCCGGGAGAGGAGGTTTTTACCAGCTATA
TTGATCTCCTGTACCCAACGGAAGATAGAAATGACCGGTTAAGAG
ATTCTTATTTCTTTACCTGTGAGTGCCAGGAGTGTACCACCAAGG
ACAAGGATAAGGCCAAGGTGGAAATCCGGAAGCTCAGCGATCCCC
CAAAGGCAGAAGCCATCCGAGACATGGTCAGATATGCACGCAACG
TCATTGAAGAGTTCCGGAGGGCCAAGCACTATAAATCCCCTAGTG
AGCTGCTGGAGATCTGCGAGCTCAGCCAGGAGAAGATGAGCTCTG
TGTTTGAGGACAGTAACGTGTACATGTTGCACATGATGTACCAGG
CCATGGGTGTCTGCTTGTACATGCAGGACTGGGAAGGAGCCCTGC
AATATGGACAGAAAATCATTAAGCCCTACAGTAAGCACTATCCTT
TGTACTCCCTCAACGTGGCCTCCATGTGGTTGAAGCTAGGGAGAC
TCTACATGGGCCTGGAACACAAAGCCGCAGGGGAGAAAGCCCTGA
AGAAGGCCATTGCAATCATGGAAGTAGCTCACGGCAAAGATCATC
CATATATTTCTGAGATCAAACAGGAAATTGAAAGCCACTGA

In certain embodiments, siRNA molecules disclosed herein may have a nucleotide sequence that is about 2-30 contiguous in length and is at least about 80% homologous (e.g., about 80%, about 85%, about 90%, about 95%, or about 99%) to at least about 2 to about 30 contiguous nucleotides of a human SMYD2 cDNA sequence. In some embodiments, siRNA molecules disclosed herein may have a nucleotide sequence that is about 2-30 contiguous in length and is at least about 80% homologous (e.g., about 80%, about 85%, about 90%, about 95%, or about 99%) to at least about 2 to about 30 contiguous nucleotides of the human SMYD2 sequence RefSeq: NM_020197.3 or a variant thereof. In some embodiments, siRNA molecules disclosed herein may have a nucleotide sequence that is about 2-30 contiguous in length and is at least about 80% homologous (e.g., about 80%, about 85%, about 90%, about 95%, or about 99%) to at least about 2 to about 30 contiguous nucleotides of SEQ ID NO: 1. In some other embodiments, siRNA molecules disclosed herein may have a nucleotide sequence that is about 2-30 contiguous in length and is homologous to at least about 2 to about 30 contiguous nucleotides of SEQ ID NO: 1.

A siRNA molecule disclosed herein may be synthesized in any number of conformations. One skilled in the art would recognize the type of siRNA conformation to be used for a particular purpose. Examples of siRNA conformations include, but need not be limited to, a double-stranded polynucleotide molecule assembled from two separate stranded molecules, wherein one strand is the sense strand and the other is the complementary antisense strand; a double-stranded polynucleotide molecule assembled from a single-stranded molecule, where the sense and antisense regions are linked by a nucleic acid-based or non-nucleic acid-based linker; a double-stranded polynucleotide molecule with a hairpin secondary structure having complementary sense and antisense regions; or a circular single-stranded polynucleotide molecule with two or more loop structures and a stem having self-complementary sense and antisense regions. In the case of the circular polynucleotide, the polynucleotide may be processed either in vivo or in vitro to generate an active double-stranded siRNA molecule.

In certain embodiments, siRNA molecules disclosed herein may be a cDNA or a double stranded siRNA molecules. In some aspects, the double stranded siRNA molecules disclosed herein may have at least one sense sequence. In some other aspects, the double stranded siRNA molecules disclosed herein may have at least one antisense sequence. In still some other aspects, double stranded siRNA molecules disclosed herein may have at least one antisense sequence and at least one sense sequence. In some embodiments, the double stranded siRNA molecules disclosed herein may have at least one antisense sequence selected from Table 1, at least one sense sequence selected from Table 1, or both.

TABLE 1

| | Seq | | Seq | | Seq | |
|---|---|---|---|---|---|---|
| SIRNA | ID | siRNA or cDNA | ID | | ID | |
| ID | No. | sequence | No. | Sense | No. | Antisense |
| SMYD | 2 | GACCGGTTAAGAG | 59 | CCGGUUAAGAGAU | 116 | AUAAGAAUCUCUU |
| 2 757 | | ATTCTTATTT | | UCUUAUUU | | AACCGGUU |
| SMYD | 3 | CACGGCAAAGATC | 60 | CGGCAAAGAUCAUC | 117 | AUAUGGAUGAUCU |
| 2 1249 | | ATCCATATAT | | CAUAUUU | | UUGCCGUU |
| SMYD | 4 | TACCCAACGGAAG | 61 | CCCAACGGAAGAUA | 118 | AUUUCUAUCUUCC |
| 2 736 | | ATAGAAATGA | | GAAAUUU | | GUUGGGUU |
| SMYD | 5 | GCCCTGCAATATG | 62 | CCUGCAAUAUGGA | 119 | UUUCUGUCCAUAU |
| 2 1078 | | GACAGAAAAT | | CAGAAAUU | | UGCAGGUU |
| SMYD | 6 | TCGGAGACTGTAA | 63 | GGAGACUGUAAGA | 120 | UGUUAGUCUUACA |
| 2 310 | | GACTAACAGC | | CUAACAUU | | GUCUCCUU |
| SMYD | 7 | CAGAGCTGTACAG | 64 | GAGCUGUACAGGA | 121 | UUGAUUUCCUGUA |
| 2 675 | | GAAATCAAGC | | AAUCAAUU | | CAGCUCUU |
| SMYD | 8 | AGCCCTGCAATAT | 65 | CCCUGCAAUAUGG | 122 | UUCUGUCCAUAUU |
| 2 1077 | | GGACAGAAAA | | ACAGAAUU | | GCAGGGUU |
| SMYD | 9 | CTCGGATTCCCTG | 66 | CGGAUUCCCUGAC | 123 | AUCAUUGUCAGGG |
| 2 493 | | ACAATGATAG | | AAUGAUUU | | AAUCCGUU |
| SMYD | 10 | ATCAGCGATATTTC | 67 | CAGCGAUAUUUCC | 124 | ACAUCAGGAAAUA |
| 2 588 | | CTGATGTTG | | UGAUGUUU | | UCGCUGUU |
| SMYD | 11 | TGCACGCAACGTC | 68 | CACGCAACGUCAUU | 125 | UCUUCAAUGACGU |
| 2 894 | | ATTGAAGAGT | | GAAGAUU | | UGCGUGUU |
| SMYD | 12 | GGCAAAGATCATC | 69 | CAAAGAUCAUCCAU | 126 | AAUAUAUGGAUGA |
| 2 1252- | | CATATATTTC | | AUAUUUC | | UCUUUGCC |
| 1274 | | | | | | |
| SMYD | 13 | GAGGTTTTTACCAG | 70 | GGUUUUUACCAGC | 127 | AAUAUAGCUGGUA |
| 2 706- | | CTATATTGA | | UAUAUUGA | | AAAACCUC |
| 728 | | | | | | |
| SMYD | 14 | CCGGTTAAGAGAT | 71 | GGUUAAGAGAUUC | 128 | AAAUAAGAAUCUCU |
| 2 759- | | TCTTATTTCT | | UUAUUUCU | | UAACCGG |
| 781 | | | | | | |
| SMYD | 15 | GAGATCAAACAGG | 72 | GAUCAAACAGGAAA | 129 | UUCAAUUUCCUGU |
| 2 1276- | | AAATTGAAAG | | UUGAAAG | | UUGAUCUC |
| 1298 | | | | | | |
| SMYD | 16 | TGGATAAGTTAGAC | 73 | GAUAAGUUAGACAA | 130 | UCUCAUUGUCUAA |
| 2 416- | | AATGAGAAG | | UGAGAAG | | CUUAUCCA |
| 438 | | | | | | |
| SMYD | 17 | TGCAATATGGACA | 74 | CAAUAUGGACAGAA | 131 | UGAUUUUCUGUCC |
| 2 1082- | | GAAAATCATT | | AAUCAUU | | AUAUUGCA |
| 1104 | | | | | | |
| SMYD | 18 | ACCGGTTAAGAGA | 75 | CGGUUAAGAGAUU | 132 | AAUAAGAAUCUCU |
| 2 758- | | TTCTTATTTC | | CUUAUUUC | | UAACCGGU |
| 780 | | | | | | |
| SMYD | 19 | GAGATTCTTATTTC | 76 | GAUUCUUAUUUCU | 133 | AGGUAAAGAAAUAA |
| 2 767- | | TTTACCTGT | | UUACCUGU | | GAAUCUC |
| 789 | | | | | | |
| SMYD | 20 | CGGCAAAGATCAT | 77 | GCAAAGAUCAUCCA | 134 | AUAUAUGGAUGAU |
| 2 1251- | | CCATATATTT | | UAUAUUU | | CUUUGCCG |
| 1273 | | | | | | |
| SMYD | 21 | TAGAAATGACCGG | 78 | GAAAUGACCGGUU | 135 | UCUCUUAACCGGU |
| 2 750- | | TTAAGAGATT | | AAGAGAUU | | CAUUUCUA |
| 772 | | | | | | |
| SMYD | 22 | GTGAAGGAGTTTG | 79 | GAAGGAGUUUGAA | 136 | AUGUGAUUCAAAC |
| 2 394 | | AATCACATCT | | UCACAUUU | | UCCUUCUU |
| SMYD | 23 | CCCTCGGAGACTG | 80 | CUCGGAGACUGUA | 137 | UAGUCUUACAGUC |
| 2 307 | | TAAGACTAAC | | AGACUAUU | | UCCGAGUU |
| SMYD | 24 | CTCTGTGTTTGAG | 81 | CUGUGUUUGAGGA | 138 | UUACUGUCCUCAA |
| 2 990 | | GACAGTAACG | | CAGUAAUU | | ACACAGUU |
| SMYD | 25 | TACCAGCTATATTG | 82 | CCAGCUAUAUUGAU | 139 | AGGAGAUCAAUAU |
| 2 714 | | ATCTCCTGT | | CUCCUUU | | AGCUGGUU |
| SMYD | 26 | TGGCTTCACAATTG | 83 | GCUUCACAAUUGAA | 140 | UCAUCUUCAAUUG |
| 2 549 | | AAGATGAAG | | GAUGAUU | | UGAAGCUU |
| SMYD | 27 | TTCACAATTGAAGA | 84 | CACAAUUGAAGAUG | 141 | UUCUUCAUCUUCA |
| 2 553 | | TGAAGAACT | | AAGAAUU | | AUUGUGUU |
| SMYD | 28 | GGCTTCACAATTGA | 85 | CUUCACAAUUGAAG | 142 | UUCAUCUUCAAUU |
| 2 550 | | AGATGAAGA | | AUGAAUU | | GUGAAGUU |
| SMYD | 29 | TCCTGTACCCAAC | 86 | CUGUACCCAACGG | 143 | UAUCUUCCGUUGG |
| 2 731 | | GGAAGATAGA | | AAGAUAUU | | GUACAGUU |
| SMYD | 30 | GGGTGTCTGCTTG | 87 | GUGUCUGCUUGUA | 144 | UGCAUGUACAAGC |
| 2 1044 | | TACATGCAGG | | CAUGCAUU | | AGACACUU |
| SMYD | 31 | CTGCAATGTGGAG | 88 | GCAAUGUGGAGUG | 145 | UUCUGACACUCCA |
| 2 222 | | TGTCAGAAAG | | UCAGAAUU | | CAUUGCUU |
| SMYD | 32 | CACGCAACGTCAT | 89 | CGCAACGUCAUUG | 146 | ACUCUUCAAUGAC |
| 2 896 | | TGAAGAGTTC | | AAGAGUUU | | GUUGCGUU |
| SMYD | 33 | CCCTGACAATGATA | 90 | CUGACAAUGAUAGC | 147 | ACGAGGCUAUCAU |
| 2 501 | | GCCTCGTAG | | CUCGUUU | | UGUCAGUU |
| SMYD | 34 | GCCCCAATGTCATT | 91 | CCCAAUGUCAUUG | 148 | AGGUCACAAUGAC |
| 2 632 | | GTGACCTAC | | UGACCUUU | | AUUGGGUU |
| SMYD | 35 | AGGAGAAGATGAG | 92 | GAGAAGAUGAGCU | 149 | ACACAGAGCUCAU |
| 2 977 | | CTCTGTGTTT | | CUGUGUUU | | CUUCUCUU |
| SMYD | 36 | ATCATCCATATATT | 93 | CAUCCAUAUAUUUC | 150 | UCUCAGAAAUAUA |
| 2 1259 | | TCTGAGATC | | UGAGAUU | | UGGAUGUU |
| SMYD | 37 | ATCCATATATTTCT | 94 | CCAUAUAUUUCUGA | 151 | UGAUCUCAGAAAU |
| 2 1262 | | GAGATCAAA | | GAUCAUU | | AUAUGGUU |
| SMYD | 38 | TTGCATTGATGAAT | 95 | GCAUUGAUGAAUCA | 152 | AGCUAUGAUUCAU |
| 2 608 | | CATAGCTGT | | UAGCUUU | | CAAUGCUU |
| SMYD | 39 | GAGACTGTAAGAC | 96 | GACUGUAAGACUAA | 153 | UGCUGUUAGUCUU |
| 2 313 | | TAACAGCAAG | | CAGCAUU | | ACAGUCUU |
| SMYD | 40 | TTGATTCAGAGTGA | 97 | GAUUCAGAGUGAC | 154 | AGCUAUGUCACUC |
| 2 445 | | CATAGCTGC | | AUAGCUUU | | UGAAUCUU |
| SMYD | 41 | AGGATAAGGCCAA | 98 | GAUAAGGCCAAGG | 155 | UUUCCACCUUGGC |
| 2 818 | | GGTGGAAATC | | UGGAAAUU | | CUUAUCUU |
| SMYD | 42 | GGGATCAGCGATA | 99 | GAUCAGCGAUAUU | 156 | UCAGGAAAUAUCG |
| 2 585 | | TTTCCTGATG | | UCCUGAUU | | CUGAUCUU |
| SMYD | 43 | CAGTAACGTGTAC | 100 | GUAACGUGUACAU | 157 | UGCAACAUGUACA |
| 2 1005 | | ATGTTGCACA | | GUUGCAUU | | CGUUACUU |
| SMYD | 44 | CTGTAAGACTAACA | 101 | GUAAGACUAACAGC | 158 | UCCUUGCUGUUAG |
| 2 317 | | GCAAGGATT | | AAGGAUU | | UCUUACUU |
| SMYD | 45 | AGGTTAACTGTAAT | 102 | GUUAACUGUAAUG | 159 | UGAAGCCAUUACA |
| 2 536 | | GGCTTCACA | | GCUUCAUU | | GUUAACUU |
| SMYD | 46 | GCGATATTTCCTGA | 103 | GAUAUUUCCUGAU | 160 | UGCAACAUCAGGA |
| 2 592 | | TGTTGCATT | | GUUGCAUU | | AAUAUCUU |
| SMYD | 47 | TGGTTGTTTTTGGG | 104 | GUUGUUUUUGGGG | 161 | AGUUUUCCCCAAA |
| 2 281- | | GAAAACTGG | | AAAACUGG | | AACAACCA |
| 303 | | | | | | |
| SMYD | 48 | GACCGGTTAAGAG | 105 | CCGGUUAAGAGAU | 162 | AUAAGAAUCUCUU |
| 2 757- | | ATTCTTATTT | | UCUUAUUU | | AACCGGUC |
| 779 | | | | | | |
| SMYD | 49 | CACAATTGAAGATG | 106 | CAAUUGAAGAUGAA | 163 | AGUUCUUCAUCUU |
| 2 555- | | AAGAACTTT | | GAACUUU | | CAAUUGUG |
| 577 | | | | | | |
| SMYD | 50 | TTCACAATTGAAGA | 107 | CACAAUUGAAGAUG | 164 | UUCUUCAUCUUCA |
| 2 553- | | TGAAGAACT | | AAGAACU | | AUUGUGAA |
| 575 | | | | | | |
| SMYD | 51 | AACTGTAATGGCTT | 108 | CUGUAAUGGCUUC | 165 | AAUUGUGAAGCCA |
| 2 541- | | CACAATTGA | | ACAAUUGA | | UUACAGUU |
| 563 | | | | | | |
| SMYD | 52 | ATGTTGCATTGATG | 109 | GUUGCAUUGAUGA | 166 | UAUGAUUCAUCAA |
| 2 605- | | AATCATAGC | | AUCAUAGC | | UGCAACAU |
| 627 | | | | | | |
| SMYD | 53 | AGGACAGTAACGT | 110 | GACAGUAACGUGU | 167 | ACAUGUACACGUU |
| 2 1001- | | GTACATGTTG | | ACAUGUUG | | ACUGUCCU |
| 1023 | | | | | | |
| SMYD | 54 | AAGGATTCTGGCC | 111 | GGAUUCUGGCCAA | 168 | UUCUGUUUGGCCA |
| 2 333- | | AAACAGAAAA | | ACAGAAAA | | GAAUCCUU |
| 355 | | | | | | |
| SMYD | 55 | CCCTGCAATATGG | 112 | CUGCAAUAUGGACA | 169 | UUUUCUGUCCAUA |
| 2 1079- | | ACAGAAAATC | | GAAAAUC | | UUGCAGGG |
| 1101 | | | | | | |
| SMYD | 56 | TTGAATCACATCTG | 113 | GAAUCACAUCUGGA | 170 | ACUUAUCCAGAUG |
| 2 404- | | GATAAGTTA | | UAAGUUA | | UGAUUCAA |
| 426 | | | | | | |
| SMYD | 57 | AAGATGAAGAACTT | 114 | GAUGAAGAACUUUC | 171 | AAUGAGAAAGUUC |
| 2 563- | | TCTCATTTG | | UCAUUUG | | UUCAUCUU |
| 585 | | | | | | |
| SMYD | 58 | GGCTTCACAATTGA | 115 | CUUCACAAUUGAAG | 172 | UUCAUCUUCAAUU |
| 2 550- | | AGATGAAGA | | AUGAAGA | | GUGAAGCC |
| 572 | | | | | | |

In some embodiments, the siRNA molecules disclosed herein may have at least one cDNA sequence having at least about 80% homology (e.g., about 80%, about 85%, about 90%, about 95%, or about 99%) to any one of SEQ ID NOs: 2-58. In some embodiments, the siRNA molecules disclosed herein may have at least one sense sequence having at least about 80% homology (e.g., about 80%, about 85%, about 90%, about 95%, or about 99%) to any one of SEQ ID NOs: 59-115. In some embodiments, double stranded siRNA molecules disclosed herein may have at least one antisense sequence having at least about 80% homology (e.g., about 80%, about 85%, about 90%, about 95%, or about 99%) to any one of SEQ ID NOs: 116-172. In some embodiments, double stranded siRNA molecules disclosed herein may have at least one sense sequence having at least about 80% homology (e.g., about 80%, about 85%, about 90%, about 95%, or about 99%) to any one of SEQ ID NOs: 59-115 and at least one antisense sequence having at least about 80% homology (e.g., about 80%, about 85%, about 90%, about 95%, or about 99%) to any one of SEQ ID NOs: 116-172. In some embodiments, the cDNA disclosed herein may have at least one sense sequence of SEQ ID NOS: 2-21. In some embodiments, double stranded siRNA molecules disclosed herein may have at least one sense sequence of SEQ ID NOs: 59-78. In some embodiments, double stranded siRNA molecules disclosed herein may have at least one antisense sequence of SEQ ID NOs: 116-135. In some embodiments, double stranded siRNA molecules disclosed herein may have at least one sense sequence of SEQ ID NOs: 59-78 and at least one antisense sequence of SEQ ID NOs: 116-135. In some aspects, the cDNA may have a sense sequence of SEQ ID NO: 9, or SEQ ID NO: 11-12. In some aspects, double stranded siRNA molecules disclosed herein may have a sense sequence of SEQ ID NO: 66, or SEQ ID NO: 68-69 and an antisense sequence of SEQ ID NO: 123, or SEQ ID NO: 125-126. In some aspect, double stranded siRNA molecules disclosed herein may have a sense sequence of SEQ ID NO: 66 and an antisense sequence of SEQ ID NO: 123. In some aspects, double stranded siRNA molecules disclosed herein may have a sense sequence of SEQ ID NO: 68 and an antisense sequence of SEQ ID NO: 125. In some aspect, double stranded siRNA molecules disclosed herein may have a sense sequence of SEQ ID NO: 69 and an antisense sequence of SEQ ID NO: 126.

The present disclosure also provides for methods of making the siRNA molecules disclosed herein. Making of siRNA molecules disclosed herein may be through chemical synthesis or siRNA molecules disclosed herein may be encoded by a plasmid and transcribed, or may be vectored by a virus engineered to express the siRNA. A siRNA molecule disclosed herein may be a single stranded molecule with complementary sequences that self-hybridize into duplexes with hairpin loops. An siRNA molecule disclosed herein can also be generated by cleavage of parent dsRNA through the use of an appropriate enzyme such as E. coli RNase III or Dicer. A parent dsRNA may be any double stranded RNA duplex from which a siRNA may be produced, such as a full or partial mRNA transcript. Use of cell lysates or in vitro processing may further involve the subsequent isolation of the short, nucleotide siRNAs (e.g., about 2-25 nucleotides in length) from the lysate, etc., making the process somewhat cumbersome and expensive. Chemical synthesis proceeds by making two single stranded RNA-oligomers followed by the annealing of the two single stranded oligomers into a double stranded RNA. Methods of chemical synthesis are diverse and can be easily adapted to synthesis different siRNAs.

In certain embodiments, siRNA molecules disclosed herein may abolish gene expression of SMYD2. Methods known in the art for the detection and quantification of RNA expression suitable for use herein can include, but are not limited to northern blotting and in situ hybridization, RNAse protection assays, polymerase chain reaction (PCR), reverse transcription polymerase chain reaction (RT-PCR), real-time quantitative reverse transcription PCR (RT-qPCR or qPCR), sequencing-based gene expression analysis (e.g., Serial Analysis of Gene Expression (SAGE)), gene expression analysis by massively parallel signature sequencing (MPSS), and the like.

The siRNA molecules of the present disclosure specifically downregulate gene expression of SMYD2 or a variant thereof. The term downregulating may be interchangeably expressed as reducing, inhibiting, preventing, blocking or silencing. Here, the phrase “downregulating gene expression” refers to any reduced level of gene expression in comparing with an ordinary expression level. For example, the reduced level of gene expression can be from at least about 70% to about 0% of the ordinary expression level. The reduced level of gene expression can be from at least about 30%, at least about 40%, at least about 50%, at least about 60%, at least about 70%, at least about 80%, or at least about 90% of ordinary SMYD2 gene expression. the ordinary expression level. In other words, about 30% to about 100% gene expression is downregulated, reduced, blocked, inhibited, prevented or silenced, comparing to the ordinary expression level. Specifically, the reduced level of gene expression is about 70%, 65%, 60%, 55%, 50%, 55%, 50%, 45%, 40%, 35%, 30%, 25%, 20%, 15%, 10%, 8%, 5%, 4%, 3%, 2%, 1% or 0% of the ordinary expression level of SMYD2. In other words, about 30% to about 100% gene expression is downregulated, reduced, blocked, inhibited, prevented or silenced, comparing to the ordinary expression level. Specifically, the reduced level of gene expression is about 70%, 65%, 60%, 55%, 50%, 55%, 50%, 45%, 40%, 35%, 30%, 25%, 20%, 15%, 10%, 8%, 5%, 4%, 3%, 2%, 1% or 0% of the ordinary expression level of SMYD2. The reduced level of gene expression can also be any percentage or range as recited above. On the other hand, the term “specific” or “specifically” used in combination with downregulating refers to downregulation of a target gene's expression with minimal or no binding or downregulation of other nucleic acids or their expressions.

In some embodiments, siRNA molecules disclosed herein may reduce gene expression of SMYD2 by at least about 50%. In some aspects, siRNA molecules disclosed herein may reduce gene expression of SMYD2 by about 50% to about 99%, about 55% to about 98%, or about 60% to about 95%. In some aspects, siRNA molecules disclosed herein may reduce gene expression of SMYD2 by about 50%, about 55%, about 60%, about 65%, about 70%, about 75%, about 80%, about 85%, about 90%, about 95%, or about 99%.

In certain embodiments, siRNA molecules disclosed herein may abolish protein expression of SMYD2. Methods known in the art for the detection and quantification of protein expression suitable for use herein can include, but are not limited to ELISAs (enzyme-linked immunosorbent assays), immunoblot assays, flow cytometric assays, immunohistochemical assays, radioimmuno assays, Western blot assays, an immunofluorescent assays, chemiluminescent assays, mass spectrometry assays, matrix-assisted laser desorption/ionization time-of-flight (MALDI-TOF) mass mapping, liquid chromatography/quadrupole time-of-flight electrospray ionization tandem mass spectrometry (LC/Q-TOF-ESI-MS/MS), two-dimensional polyacrylamide gel electrophoresis (2D-PAGE), and the like.

In some embodiments, siRNA molecules disclosed herein may reduce protein expression of SMYD2 by at least about 50%. In some aspects, siRNA molecules disclosed herein may reduce protein of SMYD2 by about 50% to about 99%, about 55% to about 98%, or about 60% to about 95%. In some other aspects, siRNA molecules disclosed herein may reduce protein expression of SMYD2 by about 50%, about 55%, about 60%, about 65%, about 70%, about 75%, about 80%, about 85%, about 90%, about 95%, or about 99%.

In some embodiments, siRNA molecules disclosed herein may have one or more chemical modifications. Non-limiting examples of chemical modifications can include terminal cap moieties, phosphate backbone modifications, and the like. Examples of classes of terminal cap moieties include, without limitation, inverted deoxy abasic residues, glyceryl modifications, 4′,5′-methylene nucleotides, 1-(β-D-erythrofuranosyl) nucleotides, 4′-thio nucleotides, carbocyclic nucleotides, 1,5-anhydrohexitol nucleotides, L-nucleotides, α-nucleotides, modified base nucleotides, threo pentofuranosyl nucleotides, acyclic 3′,4′-seco nucleotides, acyclic 3,4-dihydroxybutyl nucleotides, acyclic 3,5-dihydroxypentyl nucleotides, 3′-3′-inverted nucleotide moieties, 3′-3′-inverted abasic moieties, 3′-2′-inverted nucleotide moieties, 3′-2′-inverted abasic moieties, 5′-5′-inverted nucleotide moieties, 5′-5′-inverted abasic moieties, 3′-5′-inverted deoxy abasic moieties, 5′-amino-alkyl phosphate, 1,3-diamino-2-propyl phosphate, 3 aminopropyl phosphate, 6-aminohexyl phosphate, 1,2-aminododecyl phosphate, hydroxypropyl phosphate, 1,4-butanediol phosphate, 3′-phosphoramidate, 5′ phosphoramidate, hexylphosphate, aminohexyl phosphate, 3′-phosphate, 5′-amino, 3′-phosphorothioate, 5′-phosphorothioate, phosphorodithioate, and bridging or non-bridging methylphosphonate or 5′-mercapto moieties. Non-limiting examples of phosphate backbone modifications (i.e., resulting in modified internucleotide linkages) include phosphorothioate, phosphorodithioate, methylphosphonate, phosphotriester, morpholino, amidate, carbamate, carboxymethyl, acetamidate, polyamide, sulfonate, sulfonamide, sulfamate, formacetal, thioformacetal, and alkylsilyl substitutions. Such chemical modifications can occur at the 5′-end and/or 3′-end of the sense strand, antisense strand, or both strands of a siRNA molecule disclosed herein.

Chemical modification of a siRNA molecule disclosed herein may comprise modification of at least one ribosugar moiety of its nucleotide sequence. The ribosugar moiety may be modified with 2 2′-O-methyl (2′OMe), 2′-deoxy-2′-fluoro (2′F), 2′-deoxy, 5-C-methyl, 2′-O-(2-methoxyethyl) (MOE), 4′-thio, 2′-amino, 2′-C-allyl, or any combination thereof. In some aspects, a siRNA molecule disclosed herein may have less than about 10% to about 70% (e.g., about 10%, about 20%, about 30%, about 40%, about 50%, about 60%, about 70%) of ribosugar moieties of the total nucleotide sequence modified.

Chemical modification of a siRNA molecule disclosed herein may comprise attaching a conjugate to the siRNA molecule. The type of conjugate used and the extent of conjugation to the siRNA can be evaluated for improved pharmacokinetic profiles, bioavailability, and/or stability of the siRNA while retaining activity. As such, one skilled in the art can screen siRNA molecules having various conjugates attached thereto to identify siRNA conjugates having improved properties using any of a variety of well-known in vitro cell culture or in vivo animal models including the negative-controlled expression studies described above. The conjugate can be attached at the 5′- and/or the 3′-end of the sense and/or the antisense strand of the siRNA via a covalent attachment such as a nucleic acid or non-nucleic acid linker. The conjugate can be attached to the siRNA through a carbamate group or other linking group (see, e.g., U.S. Patent Publication Nos. 20050074771, 20050043219, and 20050158727, the content of each of which is incorporated by reference herein in its entirety). A conjugate may be added to siRNA for any of a number of purposes. For example, the conjugate may be a molecular entity that facilitates the delivery of siRNA into a cell or may be a molecule that comprises a drug or label. Examples of conjugate molecules suitable for attachment to siRNA of the present invention include, without limitation, steroids such as cholesterol, glycols such as polyethylene glycol (PEG), human serum albumin (HSA), fatty acids, carotenoids, terpenes, bile acids, folates (e.g., folic acid, folate analogs and derivatives thereof), sugars (e.g., galactose, galactosamine, N-acetyl galactosamine, glucose, mannose, fructose, fucose, etc.), phospholipids, peptides, ligands for cellular receptors capable of mediating cellular uptake, and combinations thereof. Other examples include the lipophilic moiety, vitamin, polymer, peptide, protein, nucleic acid, small molecule, oligosaccharide, carbohydrate cluster, intercalator, minor groove binder, cleaving agent, and cross-linking agent conjugate molecules described in e.g., U.S. Patent Publication Nos. 20050119470 and 20050107325, the content of each of which is incorporated by reference herein in its entirety. Other examples include the 2′-O-alkyl amine, 2′-O-alkoxyalkyl amine, polyamine, C5-cationic modified pyrimidine, cationic peptide, guanidinium group, amidininium group, cationic amino acid conjugate molecules, and the like. Additional examples of conjugate molecules include a hydrophobic group, a membrane active compound, a cell penetrating compound, a cell targeting signal, an interaction modifier, or a steric stabilizer as described in U.S. Patent Publication No. 20040167090, incorporated by reference herein in its entirety.

In certain embodiments, siRNA molecules disclosed herein may be conjugated to at least one targeting ligand. Targeting ligands contemplated herein include ligands suitable for targeting siRNA molecules to a liver, a liver tissue, and/or a liver cell. Non-limiting examples of targeting ligands suitable for use herein may include galactose, galactosamine, N-formyl-galactosamine, N-acetylgalactosamine, N-propionyl-galactosamine, N-n-butanoyl-galactosamine, N-iso-butanoylgalactos-amine, galactose cluster, and N-acetylgalactosamine trimer and may optionally have a pharmacokinetic modulator selected from the group consisting of: hydrophobic group having 16 or more carbon atoms, hydrophobic group having 16-20 carbon atoms, palmitoyl, hexadec-8-enoyl, oleyl, (9E, 12E)-octadeca-9,12dienoyl, dioctanoyl, and C16-C20 acyl, and cholesterol. In some embodiments, a liver targeting ligand suitable for use herein may be a N-Acetylgalactosamine (GalNAc) conjugate. In some embodiments, siRNA molecules disclosed herein may be conjugated to at least one GalNAc conjugate. In some embodiments, siRNA molecules disclosed herein may be conjugated to about 1 to about 10 GalNAc conjugates, about 2 to about 9 GalNAc conjugates, or about 3 to about 8 GalNAc conjugates. In some embodiments, siRNA molecules disclosed herein may be conjugated to about 1, about 2, about 3, about 4, about 5, about 6, about 7, about 8, about 9, or about 10 GalNAc conjugates.

Any of the siRNA molecules disclosed herein may target the open reading frame or the 5′ or 3′ UTRs of the SMYD2 gene or at least one variant thereof.

siRNA molecules disclosed herein can specifically downregulate gene expression of at least one variant of SMYD2. The SMYD2 gene and/or its variant may be associated with a liver disease, liver disfunction, liver injury, and/or liver damage. The liver disease may comprise fatty liver disease (FLD), alcohol-related liver disease (ARLD), non-alcoholic fatty liver disease (NAFLD), non-alcoholic steatohepatitis (NASH), end stage liver disease (cirrhosis) from any etiology, liver cancer, or any combination thereof. Liver damage may include cirrhosis, chronic infection of hepatitis B virus (HBV), chronic infection of hepatitis C virus (HCV), non-alcoholic fatty liver disease (NAFLD), non-alcoholic steatohepatitis (NASH), primary biliary cirrhosis (PBC), hereditary hemochromatosis, type 2 diabetes, obesity, tobacco use, alcohol abuse, long-term anabolic steroid use, tyrosinemia, alpha1-antitrypsin deficiency, porphyria cutanea tarda, glycogen storage diseases, Wilson disease, or any combination thereof. In some embodiments, a suitable subject for the methods herein may have or be suspected of having one or more injuries to the liver that may predispose a subject to HCC.

In certain embodiments, methods and compositions provided herein can include a vector containing any one of the siRNA molecules disclosed herein. In some embodiments, a vector for use herein can be a viral vector. As used herein, the term “viral vector” can refer to a nucleic acid vector construct that includes at least one element of viral origin and has the capacity to be packaged into a viral vector particle and encodes at least an exogenous polynucleotide. In certain embodiments, the vector and/or particle can be utilized for the purpose of transferring any nucleic acids into cells either in vitro or in vivo. Numerous viral vectors are known in the art. The term virion can refer to a single infective viral particle. “Viral vector”, “viral vector particle” and “viral particle” also refer to a complete virus particle with its DNA or RNA core and protein coat as it exists outside the cell. Non-limiting examples of viral vectors for use herein can include adenoviruses, adeno-associated viruses (AAV), herpesviruses, retroviruses, lentiviruses, integrase defective lentiviruses (IDLV), and the like. In some embodiments, a viral vector disclosed herein can be a lentiviral vector. Examples of lentiviruses include, but are not limited to, human lentiviruses such as HIV (in particular HIV-1 or HIV-2), simian immunodeficiency virus (SIV), equine infectious anemia virus (EIAV), feline immunodeficiency virus (FIV), Caprine Arthritis Encephalitis Virus (CAEV), visna and progressive pneumonia viruses of sheep, baboon pseudotype viruses, bovine immunodeficiency virus (BIV), and the like. In some embodiments, siRNA molecules and/or vectors described herein can be prepared by conventional recombinant technology known to one of skill in the art. In other embodiments, siRNA molecules and/or vectors described herein can be prepared by a gene editing methods known in the art (e.g., by CRISPR). In certain embodiments, methods provided herein can include generating a cell to express any of the siRNA molecules and/or vectors described herein. In some embodiments, vectors, viral particles, and the like as contemplated herein may be encapsulated into a liposome for delivery to a subject.

### (b) Pharmaceutical Compositions

The siRNA molecules targeting SMYD2 disclosed herein for use according to the methods described herein may be provided per se and/or as part of a pharmaceutical composition, where modulators and/or inhibitors can be mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to any of the siRNA molecules. The term “active ingredient” as used herein can also include any medium, microorganism, or cell culture wherein the siRNA molecule is synthesized, expressed and/or contained, such as a genetically modified cell, bacteria, yeast, fungus, and the culture or medium thereof.

### (i) Pharmaceutically Acceptable Carriers and Excipients

Hereinafter, the phrases “physiologically acceptable carrier” and “pharmaceutically acceptable carrier” are interchangeably used herein to refer to a carrier or a diluent that does not cause significant irritation to an organism and does not abrogate the biological activity and properties of the administered compound. An adjuvant is included under these phrases.

In certain embodiments, compositions disclosed herein may further compromise one or more pharmaceutically acceptable diluent(s), excipient(s), and/or carrier(s). As used herein, a pharmaceutically acceptable diluent, excipient, or carrier, refers to a material suitable for administration to a subject without causing undesirable biological effects or interacting in a deleterious manner with any of the components of the composition in which it is contained. Pharmaceutically acceptable diluents, carriers, and excipients can include, but are not limited to, physiological saline, Ringer's solution, phosphate solution or buffer, buffered saline, and other carriers known in the art.

In some embodiments, pharmaceutical compositions herein may also include stabilizers, anti-oxidants, colorants, other medicinal or pharmaceutical agents, carriers, adjuvants, preserving agents, stabilizing agents, wetting agents, emulsifying agents, solution promoters, salts, solubilizers, antifoaming agents, antioxidants, dispersing agents, surfactants, or any combination thereof. Herein, the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols. Techniques for formulation and administration of drugs may be found in “Remington's Pharmaceutical Sciences,” Mack Publishing Co., Easton, Pa., latest edition, which is incorporated herein by reference.

In certain embodiments, pharmaceutical compositions described herein may be formulated in conventional manner using one or more physiologically acceptable carriers comprising excipients and auxiliaries to facilitate processing of genetically modified endothelial progenitor cells into preparations which can be used pharmaceutically. In some embodiments, any of the well-known techniques, carriers, and excipients may be used as suitable and/or as understood in the art.

In certain embodiments, pharmaceutical compositions described herein may be an aqueous suspension comprising one or more polymers as suspending agents. In some embodiments, polymers that may comprise pharmaceutical compositions described herein include: water-soluble polymers such as cellulosic polymers, e.g., hydroxypropyl methylcellulose; water-insoluble polymers such as cross-linked carboxyl-containing polymers; mucoadhesive polymers, selected from, for example, carboxymethylcellulose, carbomer (acrylic acid polymer), poly(methylmethacrylate), polyacrylamide, polycarbophil, acrylic acid/butyl acrylate copolymer, sodium alginate, and dextran; or a combination thereof. In some embodiments, pharmaceutical compositions disclosed herein may comprise at least about 5%, at least about 10%, at least about 20%, at least about 25%, at least about 30%, at least about 35%, at least about 40%, at least about 45%, or at least about 50% total amount of polymers as suspending agent(s) by total weight of the composition. In some embodiments, pharmaceutical compositions disclosed herein may comprise about 5% to about 99%, about 10%, about 95%, or about 15% to about 90% total amount of polymers as suspending agent(s) by total weight of the composition.

In certain embodiments, pharmaceutical compositions disclosed herein may comprise a viscous formulation. In some embodiments, viscosity of composition herein may be increased by the addition of one or more gelling or thickening agents. In some embodiments, compositions disclosed herein may comprise one or more gelling or thickening agents in an amount to provide a sufficiently viscous formulation to remain on treated tissue. In some embodiments, pharmaceutical compositions disclosed herein may comprise at least about 5%, at least about 10%, at least about 20%, at least about 25%, at least about 30%, at least about 35%, at least about 40%, at least about 45%, or at least about 50% total amount of gelling or thickening agent(s) by total weight of the composition. In some embodiments, pharmaceutical compositions disclosed herein may comprise about 5% to about 99%, about 10%, about 95%, or about 15% to about 90% total amount of gelling or thickening agent(s) by total weight of the composition. In some embodiments, suitable thickening agents for use herein can be hydroxypropyl methylcellulose, hydroxyethyl cellulose, polyvinylpyrrolidone, carboxymethyl cellulose, polyvinyl alcohol, sodium chondroitin sulfate, sodium hyaluronate. In other aspects, viscosity enhancing agents can be acacia (gum arabic), agar, aluminum magnesium silicate, sodium alginate, sodium stearate, bladderwrack, bentonite, carbomer, carrageenan, Carbopol, xanthan, cellulose, microcrystalline cellulose (MCC), ceratonia, chitin, carboxymethylated chitosan, chondrus, dextrose, furcellaran, gelatin, Ghatti gum, guar gum, hectorite, lactose, sucrose, maltodextrin, mannitol, sorbitol, honey, maize starch, wheat starch, rice starch, potato starch, gelatin, sterculia gum, xanthum gum, gum tragacanth, ethyl cellulose, ethylhydroxyethyl cellulose, ethylmethyl cellulose, methyl cellulose, hydroxyethyl cellulose, hydroxyethylmethyl cellulose, hydroxypropyl cellulose, poly (hydroxyethyl methacrylate), oxypolygelatin, pectin, polygeline, povidone, propylene carbonate, methyl vinyl ether/maleic anhydride copolymer (PVM/MA), poly (methoxyethyl methacrylate), poly (methoxyethoxyethyl methacrylate), hydroxypropyl cellulose, hydroxypropylmethyl-cellulose (HPMC), sodium (CMC), carboxymethyl-cellulose silicon dioxide, polyvinylpyrrolidone (PVP: povidone), Splenda (dextrose, maltodextrin and sucralose), or any combination thereof.

In certain embodiments, pharmaceutical compositions disclosed herein may comprise additional agents or additives selected from a group including surface-active agents, detergents, solvents, acidifying agents, alkalizing agents, buffering agents, tonicity modifying agents, ionic additives effective to increase the ionic strength of the solution, antimicrobial agents, antibiotic agents, antifungal agents, antioxidants, preservatives, electrolytes, antifoaming agents, oils, stabilizers, enhancing agents, and the like. In some embodiments, pharmaceutical compositions disclosed herein may comprise at least about 5%, at least about 10%, at least about 20%, at least about 25%, at least about 30%, at least about 35%, at least about 40%, at least about 45%, or at least about 50% total amount of one or more agents by total weight of the composition. In some embodiments, pharmaceutical compositions disclosed herein may comprise about 5% to about 99%, about 10%, about 95%, or about 15% to about 90% total amount of one or more agents by total weight of the composition. In some embodiments, one or more of these agents may be added to improve the performance, efficacy, safety, shelf-life and/or other property of the muscarinic antagonist composition of the present disclosure. In some embodiments, additives may be biocompatible, without being harsh, abrasive, and/or allergenic.

In certain embodiments, pharmaceutical compositions disclosed herein may comprise one or more acidifying agents. As used herein, “acidifying agents” refers to compounds used to provide an acidic medium. Such compounds include, by way of example and without limitation, acetic acid, amino acid, citric acid, fumaric acid and other alpha hydroxy acids, such as hydrochloric acid, ascorbic acid, and nitric acid and others known to those of ordinary skill in the art. In some embodiments, any pharmaceutically acceptable organic or inorganic acid may be used. In some embodiments, pharmaceutical compositions disclosed herein may comprise at least about 5%, at least about 10%, at least about 20%, at least about 25%, at least about 30%, at least about 35%, at least about 40%, at least about 45%, at least about 50% total amount of one or more acidifying agents by total weight of the composition.

In some embodiments, pharmaceutical compositions disclosed herein may comprise about 5% to about 99%, about 10%, about 95%, or about 15% to about 90% total amount of one or more acidifying agents by total weight of the composition.

In certain embodiments, pharmaceutical compositions disclosed herein may comprise one or more alkalizing agents. As used herein, “alkalizing agents” are compounds used to provide alkaline medium. Such compounds include, by way of example and without limitation, ammonia solution, ammonium carbonate, diethanolamine, monoethanolamine, potassium hydroxide, sodium borate, sodium carbonate, sodium bicarbonate, sodium hydroxide, triethanolamine, and trolamine and others known to those of ordinary skill in the art. In some embodiments, any pharmaceutically acceptable organic or inorganic base can be used. In some embodiments, pharmaceutical compositions disclosed herein may comprise at least about 5%, at least about 10%, at least about 20%, at least about 25%, at least about 30%, at least about 35%, at least about 40%, at least about 45%, at least about 50% total amount of one or more alkalizing agents by total weight of the composition. In some embodiments, pharmaceutical compositions disclosed herein may comprise about 5% to about 99%, about 10%, about 95%, or about 15% to about 90% total amount of one or more alkalizing agents by total weight of the composition.

In certain embodiments, pharmaceutical compositions disclosed herein may comprise one or more antioxidants. As used herein, “antioxidants” are agents that inhibit oxidation and thus can be used to prevent the deterioration of preparations by the oxidative process. Such compounds include, by way of example and without limitation, ascorbic acid, ascorbyl palmitate, butylated hydroxyanisole, butylated hydroxytoluene, hypophophorous acid, monothioglycerol, propyl gallate, sodium ascorbate, sodium bisulfite, sodium formaldehyde sulfoxylate, sodium metabisulfite and other materials known to one of ordinary skill in the art. In some embodiments, pharmaceutical compositions disclosed herein may comprise at least about 5%, at least about 10%, at least about 20%, at least about 25%, at least about 30%, at least about 35%, at least about 40%, at least about 45%, at least about 50% total amount of one or more antioxidants by total weight of the composition. In some embodiments, pharmaceutical compositions disclosed herein may comprise about 5% to about 99%, about 10%, about 95%, or about 15% to about 90% total amount of one or more antioxidants by total weight of the composition.

In certain embodiments, pharmaceutical compositions disclosed herein may comprise a buffer system. As used herein, a “buffer system” is a composition comprised of one or more buffering agents wherein “buffering agents” are compounds used to resist change in pH upon dilution or addition of acid or alkali. Buffering agents include, by way of example and without limitation, potassium metaphosphate, potassium phosphate, monobasic sodium acetate and sodium citrate anhydrous and dihydrate and other materials known to one of ordinary skill in the art. In some embodiments, any pharmaceutically acceptable organic or inorganic buffer can be used. In some embodiments, pharmaceutical compositions disclosed herein may comprise at least about 5%, at least about 10%, at least about 20%, at least about 25%, at least about 30%, at least about 35%, at least about 40%, at least about 45%, at least about 50% total amount of one or more buffering agents by total weight of the composition. In some embodiments, pharmaceutical compositions disclosed herein may comprise about 5% to about 99%, about 10%, about 95%, or about 15% to about 90% total amount of one or more buffering agents by total weight of the composition.

In some embodiments, the amount of one or more buffering agents may depend on the desired pH level of a composition. In some embodiments, pharmaceutical compositions disclosed herein may have a pH of about 6 to about 9. In some embodiments, pharmaceutical compositions disclosed herein may have a pH greater than about 8, greater than about 7.5, greater than about 7, greater than about 6.5, or greater than about 6.

In certain embodiments, pharmaceutical compositions disclosed herein may comprise one or more preservatives. As used herein, “preservatives” refers to agents or combination of agents that inhibits, reduces or eliminates bacterial growth in a pharmaceutical dosage form. Non-limiting examples of preservatives include Nipagin, Nipasol, isopropyl alcohol and a combination thereof. In some embodiments, any pharmaceutically acceptable preservative can be used. In some embodiments, pharmaceutical compositions disclosed herein may comprise at least about 5%, at least about 10%, at least about 20%, at least about 25%, at least about 30%, at least about 35%, at least about 40%, at least about 45%, at least about 50% total amount of one or more preservatives by total weight of the composition. In some embodiments, pharmaceutical compositions disclosed herein may comprise about 5% to about 99%, about 10%, about 95%, or about 15% to about 90% total amount of one or more preservatives by total weight of the composition.

In certain embodiments, pharmaceutical compositions disclosed herein may comprise one or more surface-acting reagents or detergents. In some embodiments, surface-acting reagents or detergents may be synthetic, natural, or semi-synthetic. In some embodiments, compositions disclosed herein may comprise anionic detergents, cationic detergents, zwitterionic detergents, ampholytic detergents, amphoteric detergents, nonionic detergents having a steroid skeleton, or a combination thereof. In some embodiments, pharmaceutical compositions disclosed herein may comprise at least about 5%, at least about 10%, at least about 20%, at least about 25%, at least about 30%, at least about 35%, at least about 40%, at least about 45%, at least about 50% total amount of one or more surface-acting reagents or detergents by total weight of the composition. In some embodiments, pharmaceutical compositions disclosed herein may comprise about 5% to about 99%, about 10%, about 95%, or about 15% to about 90% total amount of one or more surface-acting reagents or detergents by total weight of the composition.

In certain embodiments, pharmaceutical compositions disclosed herein may comprise one or more stabilizers. As used herein, a “stabilizer” refers to a compound used to stabilize an active agent against physical, chemical, or biochemical process that would otherwise reduce the therapeutic activity of the agent. Suitable stabilizers include, by way of example and without limitation, succinic anhydride, albumin, sialic acid, creatinine, glycine and other amino acids, niacinamide, sodium acetyltryptophonate, zinc oxide, sucrose, glucose, lactose, sorbitol, mannitol, glycerol, polyethylene glycols, sodium caprylate and sodium saccharin and others known to those of ordinary skill in the art. In some embodiments, pharmaceutical compositions disclosed herein may comprise at least about 5%, at least about 10%, at least about 20%, at least about 25%, at least about 30%, at least about 35%, at least about 40%, at least about 45%, at least about 50% total amount of one or more stabilizers by total weight of the composition. In some embodiments, pharmaceutical compositions disclosed herein may comprise about 5% to about 99%, about 10%, about 95%, or about 15% to about 90% total amount of one or more stabilizers by total weight of the composition.

In some embodiments, pharmaceutical compositions disclosed herein may comprise one or more tonicity agents. As used herein, a “tonicity agents” refers to a compound that can be used to adjust the tonicity of the liquid formulation. Suitable tonicity agents include, but are not limited to, glycerin, lactose, mannitol, dextrose, sodium chloride, sodium sulfate, sorbitol, trehalose and others known to those or ordinary skill in the art. Osmolarity in a composition may be expressed in milliosmoles per liter (mOsm/L). Osmolarity may be measured using methods commonly known in the art. In some embodiments, a vapor pressure depression method is used to calculate the osmolarity of the compositions disclosed herein. In some embodiments, the amount of one or more tonicity agents comprising a pharmaceutical composition disclosed herein may result in a composition osmolarity of about 150 mOsm/L to about 500 mOsm/L, about 250 mOsm/L to about 500 mOsm/L, about 250 mOsm/L to about 350 mOsm/L, about 280 mOsm/L to about 370 mOsm/L or about 250 mOsm/L to about 320 mOsm/L. In some embodiments, a composition herein may have an osmolality ranging from about 100 mOsm/kg to about 1000 mOsm/kg, from about 200 mOsm/kg to about 800 mOsm/kg, from about 250 mOsm/kg to about 500 mOsm/kg, or from about 250 mOsm/kg to about 320 mOsm/kg, or from about 250 mOsm/kg to about 350 mOsm/kg or from about 280 mOsm/kg to about 320 mOsm/kg. In some embodiments, a pharmaceutical composition described herein may have an osmolarity of about 100 mOsm/L to about 1000 mOsm/L, about 200 mOsm/L to about 800 mOsm/L, about 250 mOsm/L to about 500 mOsm/L, about 250 mOsm/L to about 350 mOsm/L, about 250 mOsm/L to about 320 mOsm/L, or about 280 mOsm/L to about 320 mOsm/L. In some embodiments, pharmaceutical compositions disclosed herein may comprise at least about 5%, at least about 10%, at least about 20%, at least about 25%, at least about 30%, at least about 35%, at least about 40%, at least about 45%, at least about 50% total amount of one or more tonicity modifiers by total weight of the composition. In some embodiments, pharmaceutical compositions disclosed herein may comprise about 5% to about 99%, about 10%, about 95%, or about 15% to about 90% total amount of one or more tonicity modifiers by total weight of the composition.

### (ii) Dosage Formulations

In certain embodiments, the present disclosure provides compositions formulated for one or more routes of administration. Suitable routes of administration may, for example, include oral, rectal, transmucosal, transnasal, intestinal, and/or parenteral delivery. In some embodiments, compositions herein formulated can be formulated for parenteral delivery. In some embodiments, compositions herein formulated can be formulated intramuscular, subcutaneous, intramedullary, intravenous, intraperitoneal, and/or intranasal injections.

In certain embodiments, one may administer a composition herein in a local or systemic manner, for example, via local injection of the pharmaceutical composition directly into a tissue region of a patient. In some embodiments, a pharmaceutical composition disclosed herein can be administered parenterally, e.g., by intravenous injection, intracerebroventricular injection, intra-cisterna magna injection, intra-parenchymal injection, or a combination thereof. In some embodiments, a pharmaceutical composition disclosed herein can administered to subject as disclosed herein. In some embodiments, a pharmaceutical composition disclosed herein can administered to human patient. In some embodiments, a pharmaceutical composition disclosed herein can administered to a human patient via at least two administration routes. In some embodiments, the combination of administration routes by be intracerebroventricular injection and intravenous injection; intrathecal injection and intravenous injection; intra-cisterna magna injection and intravenous injection; and/or intra-parenchymal injection and intravenous injection.

In certain embodiments, pharmaceutical compositions of the present disclosure may be manufactured by processes well known in the art, e.g., by means of conventional mixing, dissolving, granulating, dragee-making, levigating, emulsifying, encapsulating, entrapping or lyophilizing processes.

In certain embodiments, pharmaceutical compositions for use in accordance with the present disclosure thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen. For injection, the active ingredients of a pharmaceutical composition herein may be formulated in aqueous solutions, preferably in physiologically compatible buffers such as Hank's solution, Ringer's solution, physiological salt buffer, or any combination thereof.

In certain embodiments, pharmaceutical compositions described herein may be formulated in the form of a nanoparticle. The nanoparticle may have a monolayer enclosing the nanoparticle core, wherein the siRNA molecule is disposed within the nanoparticle core. In an embodiment, the nanoparticle core includes a solid lipid (i.e., lipid that remains solid at room temperature and body temperature) or a liquid lipid (i.e., oil, which remains liquid at room temperature and body temperature, for example, vegetable oil or a lipid extracted from human adipose tissue). In particular, embodiments of the present disclosure include nanoparticles and compositions for the controlled and/or sustained release (e.g., release at a predetermined rate to maintain a certain concentration for a certain period of time) of an agent, such as a small interfering RNA (siRNA) from the nanoparticle.

In certain embodiments, pharmaceutical compositions described herein may be formulated for parenteral administration, e.g., by bolus injection or continuous infusion. Formulations for injection herein may be presented in unit dosage form, e.g., in ampoules or in multidose containers with optionally, an added preservative. In some embodiments, compositions herein may be suspensions, solutions or emulsions in oily or aqueous vehicles, and/or may contain formulatory agents such as suspending, stabilizing and/or dispersing agents.

In certain embodiments, pharmaceutical compositions herein formulated for parenteral administration may include aqueous solutions of the active preparation (e.g., a siRNA molecule) in water-soluble form. In some embodiments, compositions herein comprising suspensions of the active preparation may be prepared as oily or water-based injection suspensions. Suitable lipophilic solvents and/or vehicles for use herein may include, but are not limited to, fatty oils such as sesame oil, or synthetic fatty acids esters such as ethyl oleate, triglycerides or liposomes. In some embodiments, compositions herein comprising aqueous injection suspensions may contain substances which increase the viscosity of the suspension, such as sodium carboxymethyl cellulose, sorbitol, and/or dextran. In some embodiments, compositions herein comprising a suspension may also contain one or more suitable stabilizers and/or agents which increase the solubility of the active ingredients (e.g., a siRNA molecule) to allow for the preparation of highly concentrated solutions.

In some embodiments, compositions herein may comprise the active ingredient in a powder form for constitution with a suitable vehicle, e.g., sterile, pyrogen-free water-based solution, before use.

Pharmaceutical compositions suitable for use in context of the present disclosure may include compositions wherein the active ingredients can be contained in an amount effective to achieve the intended purpose. In some embodiments, a therapeutically effective amount means an amount of active ingredients (e.g., a siRNA molecule) effective to prevent, slow, alleviate or ameliorate symptoms of a disorder (e.g., lymphoproliferative disorders, lymphoid malignancy) or prolong the survival of the subject being treated. Determination of a therapeutically effective amount is well within the capability of those skilled in the art, especially in light of the detailed disclosure provided herein. For any preparation used in the methods of the present disclosure, the therapeutically effective amount or dose can be estimated initially from in vitro and cell culture assays and or screening platforms disclosed herein. For example, a dose can be formulated in animal models to achieve a desired concentration or titer. Such information can be used to more accurately determine useful doses in humans.

In some embodiments, toxicity and therapeutic efficacy of the active ingredients disclosed herein (e.g., a siRNA molecule) can be determined by standard pharmaceutical procedures in vitro, in cell cultures or experimental animals. In some embodiments, data obtained from these in vitro and cell culture assays and animal studies can be used in formulating a range of dosage for use in a human subject. In some embodiments, a dosage for use herein may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1).

In certain embodiments, dosage amounts and/or dosing intervals may be adjusted individually to brain or blood levels of the active ingredient that are sufficient to induce or suppress the biological effect (minimal effective concentration, MEC). In some embodiments, the MEC for an active ingredient (e.g., a siRNA molecule or composition disclosed herein) may vary for each preparation but can be estimated from in vitro data. In some embodiments, dosages necessary to achieve the MEC herein may depend on individual characteristics and route of administration. Detection assays can be used to determine plasma concentrations.

In certain embodiments, depending on the severity and responsiveness of the condition to be treated, dosing with compositions herein can be of a single or a plurality of administrations, with course of treatment lasting from several days to several weeks or until cure is effected or diminution of the disease state is achieved.

In certain embodiments, amounts of a composition herein to be administered will be dependent on the subject being treated, the severity of the affliction, the manner of administration, the judgment of the prescribing physician, and the like. In some embodiments, effective doses may be extrapolated from dose-responsive curves derived from in vitro or in vivo test systems.

### III. Methods

The present disclosure provides for methods of treating, attenuating, and preventing liver disease in a subject in need thereof. In several embodiments, a method for treating, attenuating, or preventing liver disease in a subject can include administering to a subject, including a human subject, an effective amount of one or more siRNA molecules targeting SMYD2 disclosed herein.

Methods disclosed herein may include treating a subject in need thereof by administrating a therapeutically effective amount of one or more siRNA molecules or a pharmaceutical composition disclosed herein. The subject may be a human subject having or suspected of having, or at risk of having liver disease, liver damage, liver dysfunction, liver injury. The term “liver disease”, “liver injury” or “liver dysfunction” may be used interchangeably and refer to any injury of the liver, including but not limited to hardening of the liver, scarring of the liver, decreased or abnormal biliary tract function, abnormal liver enzyme activity, cirrhosis of the liver, abnormal physiology as determined by common diagnostic methods include but not limited to ultrasound, or biopsy/histopathology, necrosis of the liver and the like. Non-limiting examples of liver disease to be treated using the methods disclosed herein may include fatty liver disease (FLD), alcohol-related liver disease (ARLD), non-alcoholic fatty liver disease (NAFLD), non-alcoholic steatohepatitis (NASH), end stage liver disease (cirrhosis) from any etiology, liver cancer HCC, or any combination thereof. In some embodiments, the subject benefits of an increase in life expectancy compared to an untreated subject with identical disease condition and predicted outcome. In some other embodiments, the treatment improves the subject's liver function as compared to an untreated subject with identical disease condition and predicted outcome. In yet some other embodiments, the treatment attenuates the subject's liver fibrosis as compared to an untreated subject with identical disease condition and predicted outcome. In some embodiments, the treatment prevents additional liver fibrosis in the subject compared to an untreated subject with identical disease condition and predicted outcome.

A subject suitable for the liver disease treatment as disclosed herein may be selected based on the subject's diagnosis. In some embodiments, a method of diagnosis may detect one or more serum markers indicative of liver disease. Non-limiting examples of serum markers indicative of a liver disease (e.g., NAFLD, NASH or HCC) may include alpha-fetoprotein (AFP) (e.g., an AFP level of 20 ng/ml or higher), des-gamma-carboxy prothrombin, lens culinaris agglutinin-reactive AFP (AFP-L3), and the like. The diagnosis method may also include the evaluation of at least one clinical symptom associated with a liver disease. Non-limiting examples of clinical symptoms associated with a liver disease may include mild to moderate upper abdominal pain, weight loss, early satiety, or a palpable mass in the upper abdomen, paraneoplastic syndrome, hypoglycemia, erythrocytosis, hypercalcemia, intractable diarrhea and associated electrolyte disturbances (e.g., hyponatremia, hypokalemia, metabolic alkalosis), cutaneous manifestations (e.g., dermatomyositis, pemphigus foliaceus, seborrheic keratosis, pityriasis rotunda), intraperitoneal bleeding, jaundice, fever, pyogenic liver abscess, and the like. Other aspects of diagnosis may include at diagnosis and/or a determination of severity of cirrhosis, chronic infection of hepatitis B virus (HBV), chronic infection of hepatitis C virus (HCV), non-alcoholic fatty liver disease (NAFLD), non-alcoholic steatohepatitis (NASH), primary biliary cirrhosis (PBC), hereditary hemochromatosis, type 2 diabetes, obesity, tobacco use, alcohol abuse, long-term anabolic steroid use, tyrosinemia, alpha1-antitrypsin deficiency, porphyria cutanea tarda, glycogen storage diseases, Wilson disease, or any combination thereof.

In some embodiments, a subject can be diagnosed and/or predicted to have high or low risk for a liver disease (e.g., NASH or NAFLD) by histological or imaging-based examinations, such as contrast-enhanced multiphase CT, ultrasound, and/or MRI. Imaging features used to diagnose may include liver size, kinetics, and pattern of contrast enhancement, and growth on serial imaging wherein size may be measured as the maximum cross-section diameter on the image where the lesion is most clearly seen. The histologic appearance of NASH or NAFLD biopsies can include steatosis, inflammation, and fibrosis.

The methods and compositions of the present disclosure are useful for the treatment of subjects having fatty liver related disorders, such as NAFLD and/or NASH. The subject may have normal or substantially normal biliary tract function. Normal or substantially normal biliary tract function may be determined in a subject using any suitable methods known in the art. Generally, preferred tests for biliary tract function in NASH patients may be characterized in two groups: physiological based tests and biochemical based tests. Physiological based tests may include but are not limited to abdominal ultrasound, abdominal CT scan, endoscopic retrograde cholangiopancreatography (ECRP), Percutaneous transhepatic cholangiogram (PTCA) or Magnetic resonance cholangiopancreatography (MRCP). Biochemical based tests may include but are not limited to GGT tests, liver function tests, bilirubin tests, alkaline phosphatase (ALP) tests, liver enzyme tests, amylase blood test, lipase blood test, prothrombin time, and measurement of urine bilirubin. In some cases, one or more tests may be used to characterize biliary function. In some cases, a combination of tests may be used to assess biliary function in NASH subjects.

In some embodiments, any of the methods disclosed herein can further include monitoring for an occurrence of one or more adverse effects in the subject. Adverse effects may include, but are not limited to, hepatic impairment, hematologic toxicity, neurologic toxicity, cutaneous toxicity, gastrointestinal toxicity, or a combination thereof. When one or more adverse effects are observed, the methods disclosed herein can further include reducing or increasing the dose of one or more of the treatment regimens depending on the adverse effect or effects in the subject. For example, when a moderate to severe hepatic impairment is observed in a subject after treatment, compositions of use to treat the subject can be reduced in concentration or frequency.

In certain embodiments, treatments administered according to the methods disclosed herein can improve patient life expectancy compared to the life expectancy of an untreated subject with identical disease condition (e.g., NAFLD or NASH) and predicted outcome. As used herein, “patient life expectancy” is defined as the time at which 50 percent of subjects are alive and 50 percent have passed away. In some embodiments, patient life expectancy can be indefinite following treatment according to the methods disclosed herein. In other aspects, patient life expectancy can be increased at least about 5% or greater to at least about 100%, at least about 10% or greater to at least about 95% or greater, at least about 20% or greater to at least about 80% or greater, at least about 40% or greater to at least about 60% or greater compared to an untreated subject with identical disease condition and predicted outcome. In some embodiments, patient life expectancy can be increased at least about 5% or greater, at least about 10% or greater, at least about 15% or greater, at least about 20% or greater, at least about 25% or greater, at least about 30% or greater, at least about 35% or greater, at least about 40% or greater, at least about 45% or greater, at least about 50% or greater, at least about 55% or greater, at least about 60% or greater, at least about 65% or greater, at least about 70% or greater, at least about 75% or greater, at least about 80% or greater, at least about 85% or greater, at least about 90% or greater, at least about 95% or greater, at least about 100% compared to an untreated subject with identical disease condition and predicted outcome. In some embodiments, patient life expectancy can be increased at least about 5% or greater to at least about 10% or greater, at least about 10% or greater to at least about 15% or greater, at least about 15% or greater to at least about 20% or greater, at least about 20% or greater to at least about 25% or greater, at least about 25% or greater to at least about 30% or greater, at least about 30% or greater to at least about 35% or greater, at least about 35% or greater to at least about 40% or greater, at least about 40% or greater to at least about 45% or greater, at least about 45% or greater to at least about 50% or greater, at least about 50% or greater to at least about 55% or greater, at least about 55% or greater to at least about 60% or greater, at least about 60% or greater to at least about 65% or greater, at least about 65% or greater to at least about 70% or greater, at least about 70% or greater to at least about 75% or greater, at least about 75% or greater to at least about 80% or greater, at least about 80% or greater to at least about 85% or greater, at least about 85% or greater to at least about 90% or greater, at least about 90% or greater to at least about 95% or greater, at least about 95% or greater to at least about 100% compared to an untreated patient with identical disease condition and predicted outcome.

In some embodiments, treatment of a liver disease, such as NAFLD or NASH, according to the methods disclosed herein can result in an attenuating, a shrinking, a reducing or a preventing of a liver fibrosis in comparison to the starting size of the liver fibrosis. In some embodiments, liver fibrosis attenuating or shrinking may be at least about 5% or greater to at least about 10% or greater, at least about 10% or greater to at least about 15% or greater, at least about 15% or greater to at least about 20% or greater, at least about 20% or greater to at least about 25% or greater, at least about 25% or greater to at least about 30% or greater, at least about 30% or greater to at least about 35% or greater, at least about 35% or greater to at least about 40% or greater, at least about 40% or greater to at least about 45% or greater, at least about 45% or greater to at least about 50% or greater, at least about 50% or greater to at least about 55% or greater, at least about 55% or greater to at least about 60% or greater, at least about 60% or greater to at least about 65% or greater, at least about 65% or greater to at least about 70% or greater, at least about 70% or greater to at least about 75% or greater, at least about 75% or greater to at least about 80% or greater, at least about 80% or greater to at least about 85% or greater, at least about 85% or greater to at least about 90% or greater, at least about 90% or greater to at least about 95% or greater, at least about 95% or greater to at least about 100% (meaning that the liver fibrosis is completely gone after treatment) compared to the starting size of the liver fibrosis.

In some embodiments, treatment of a liver disease, such as NAFLD or NASH, according to the methods disclosed herein can result in an improved liver function. Liver function may be tested through routine biochemical methods. Biochemical based tests may include but are not limited to GGT tests, liver function tests, bilirubin tests, alkaline phosphatase (ALP) tests, liver enzyme tests, amylase blood test, lipase blood test, prothrombin time, and measurement of urine bilirubin. In some cases, one or more tests may be used to characterize biliary function. In some cases, a combination of tests may be used to assess biliary functions. The liver function improvement can also be assessed by subject's symptom relief, such as relieving in fatigue, weight loss, and weakness of the subject. Other relieved symptoms may include reduced fluid retention, muscle wasting, bleeding from the intestines, and any combination thereof.

### IV. Kits

The present disclosure provides kits for use in the methods disclosed herein. In some aspects, the present disclosure provides a kit for treating a liver disease (e.g., NASH or NAFLD) as disclosed herein and/or for diagnosing the liver disease. Such a kit may comprise a means for holding and/or administering such a siRNA composition or a pharmaceutical composition.

In some embodiments, kits disclosed herein can have a medical container, which holds the composition in a safe, stable and durable way. In some examples, kits disclosed herein may also comprise a means to administer the composition, such as a needle or a spatula.

Any of the kits may further comprise an instruction manual providing guidance for using the kit for treatment. The manual may be written with the physician or the liver specialist as the intended reader.

Having described several embodiments, it will be recognized by those skilled in the art that various modifications, alternative constructions, and equivalents may be used without departing from the spirit of the present inventive concept. Additionally, a number of well-known processes and elements have not been described in order to avoid unnecessarily obscuring the present inventive concept. Accordingly, this description should not be taken as limiting the scope of the present inventive concept.

Those skilled in the art will appreciate that the presently disclosed embodiments teach by way of example and not by limitation. Therefore, the matter contained in this description or shown in the accompanying drawings should be interpreted as illustrative and not in a limiting sense. The following claims are intended to cover all generic and specific features described herein, as well as all statements of the scope of the method and assemblies, which, as a matter of language, might be said to fall there between.

### EXAMPLES

The following examples are included to demonstrate preferred embodiments of the disclosure. It should be appreciated by those of skill in the art that the techniques disclosed in the examples that follow represent techniques discovered by the inventor to function well in the practice of the present disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the present disclosure.

### Example 1. Study Design

Somatic mutations are common in most individuals, and there is accumulating evidence that mutation burden increases with age and chronic tissue damage. It is unclear how somatic mutations contribute to organismal aging or disease pathogenesis, and more specifically, whether or not somatic mutations can cause a reversal or adaptation to disease. In liver tissues from NASH patients, it was recently identified recurrent and convergent mutations in genes central to insulin signaling and lipogenesis. The detection of loss of function mutations in metabolic enzymes that generate hepatic lipids suggest that some somatic mutations can confer increased fitness through a reversal of the driving etiology of disease. While these genetic alterations are presumed to be positively selected by virtue of their ability to counteract disease pathogenesis, functional evidence is still required.

To understand the biological importance of somatic mutations at the cellular, tissue, and organismal levels, mouse models that are capable of replicating a high density of mutations in the context of common liver diseases were developed. In these studies, the introduction of perturbations in individual genes essential for driving NASH revealed that decreased lipid accumulation can promote clonal fitness and expansion in NASH. Furthermore, the fate of mutant clones were evaluated in a parallel, pooled fashion within normal and NASH livers. Remarkably, in vivo screening of NASH candidate genes revealed that the somatic mutations detected in human liver tissues were also the most positively selected in mouse models of fatty liver, but were not selected for in the absence of disease. Mechanistically, these mutations reverse lipotoxic phenotypes to increase the survival of hepatocyte clones. These findings uncovered the biological basis for positive selection of somatic mutations in NASH patient livers. Based on these observations, it was reasoned that identifying mutant cells with greater fitness than wild-type (WT) cells within diseased environments might nominate new therapeutic targets. These finding encouraged to explore genes beyond those that are known to be somatically mutated by performing additional in vivo CRISPR screens for genes that are dysregulated in chronic liver disease. These screenings identified genes that when inhibited, promote liver fitness through the mitigation of lipotoxicity. The present disclosure proposed that evolutionary selection in somatically mosaic tissues is a new approach for the identification of adaptive metabolic disease pathways and therapeutic targets.

Recent evidence from the sequencing of human liver disease samples suggested that mutations could be adaptive. Research indicated that some mutations in cirrhotic livers can result in the proliferative expansion of regenerative clones in the context of chemically induced liver injuries, however, it is unclear if these expansion events protect against clinically relevant causes of liver disease. With increasing over-nutrition and obesity, non-alcoholic steatohepatitis (NASH) was rapidly becoming the leading cause of liver disease in the world. The excessive accumulation of lipid droplets in hepatocytes eventually may lead to lipotoxicity, cell death, and cirrhosis. NASH is usually conceptualized at the organismal and tissue levels, and little thought has been given to genetic heterogeneity between clones in the liver. Chronic injury and age increase the abundance of mutant clones in solid organs such as the liver. The present disclosure presented studies conducted to uncover the positive selection of somatically mutated clones related to the adaptive pathways in metabolic liver disease. The studies were appropriately dominated by deep sequencing that have statistically inferred fitness changes based on recurrency and clone size of somatic mutations. The present disclosure addressed multiple questions on the functional understanding of how mutant clones influence fitness on different biological levels (cell, tissue, organism). These addressed questions included if human genetic findings can be modeled and validated functionally; if mutations that benefit small cellular clones can also benefit the tissue or organism, and if somatic mutations can be used to identify adaptive genetic changes that might inspire therapeutic strategies. These questions were answered by a series of studies. First, clones with mutations in important NASH genes were lineage-traced, demonstrating the increased lipid accumulation is detrimental to mosaic clone survival in livers afflicted with NASH. To assess mutant clones in a massively parallel fashion, a new in vivo screening platform called MOSAICS was established. The advance associated with the MOSAICS platform is that it allows for in vivo screening with a 50-fold greater gene density, and in a non-proliferative setting. These approaches allowed to validate the functional importance of key somatic genetic discoveries in metabolic liver disease, such as mutations in SMYD2.

### Example 2. Results

### Analysis of Clonal Evolution in Somatically Mutated Livers with and without Steatosis

To understand how mutations in different genes might influence clonal dynamics in an unbiased fashion, a platform called MOSAICS (Method Of Somatic AAV-transposon In vivo Clonal Screening) was developed, which was a CRISPR based method to generate pools of heterogeneous mutant cells within tissues such as the liver. MOSAICS was completely distinct from previous Fah KO based regeneration screening systems because it is designed to assess a much higher density of mutant clones during homeostasis, and does not require rapid proliferation based selection of FAH expressing clones.

While AAVs were optimal for use in the liver, traditional AAVs cannot be used for screening because they do not genomically integrate, and thus their sgRNAs cannot be later quantified. The MOSAICS AAV vector carried a U6 promoter driven sgRNA element and a CAG promoter driven Sleeping Beauty 100 transposase (SB100)-P2A-Cre fusion protein. The entire AAV payload is flanked by transposon inverted repeat (IR) sequences that enable genome integration of the payload in the presence of SB100 protein, and thus enables long-term tracing of integrated sgRNAs. Prior studies have combined AAVs with transposons, but components within the MOSAICS vector and AAV were specifically engineered to meet the needs of generating and tracing mutations in the liver. The dose dependent effects of a MOSAICS vector were assessed containing an sgRNA against Pten. The MOSAICS AAV8-sgPten were injected into doxycycline (dox)-inducible TetO-Cas9 mice to generate somatic mutations in the liver, or into LSL-tdTomato mice to monitor the expression of SB100-P2A-Cre protein, which activated Tomato. Increasing amounts of AAV could delete Pten or activate the Tomato reporter in an increasing number of hepatocytes, indicating proper functioning of the vector elements and that this system could be titrated to generate different levels of somatic mosaicism, or alternatively, liver-wide gene deletion.

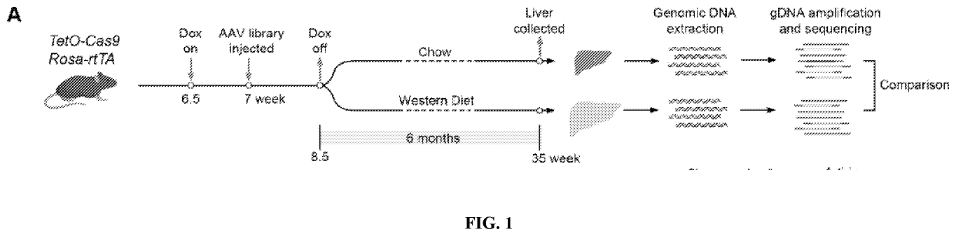

After validation of the MOSAICS system, the next aim was to determine if genes known to be important in NASH would have substantial effects on clonal fitness. An sgRNA library were generated against 63 NASH genes. Mosaically mutated livers were generated by injecting the AAV library into Cas9 expressing mice, and then exposed the mice to either normal chow or WD. Deep sequencing and analysis of enriched or depleted guides after a fixed time period enabled us to monitor clonal dynamics in both chow and WD conditions (FIG. 1). By including sgRNAs specifically enriched in WD conditions and excluding sgRNAs enriched in both chow and WD conditions, we were able to identify fatty liver specific, fitness promoting mutations and exclude genes whose mutations induce a general, fatty liver independent proliferation. Seven to eight independent mice were used for each group, and the results from individual mice were largely consistent. While liver weight, body weight, and steatosis increased as expected on WD, surface liver tumors were not detected in mice fed with WD for 6 months, suggesting that the somatic mutations did not cause rapid cancer development.

### Somatic Mosaic Screening of Candidate Genes Identifies Smyd2 as a Therapeutic Target for NASH

Positive selection of specific clones in the NASH environment suggested that some somatic mutations can promote fitness through altered lipid accumulation or resistance to lipotoxicity. Therefore, clone size/sgRNA abundance can be a surrogate measure for a gene's influence on cellular fitness in NASH livers. Based on fitness competition, MOSAICS represents an entirely new way of identifying NASH genes. Equipped with this novel method, it was sought to discover new functionally important genes and fitness-promoting mutations in

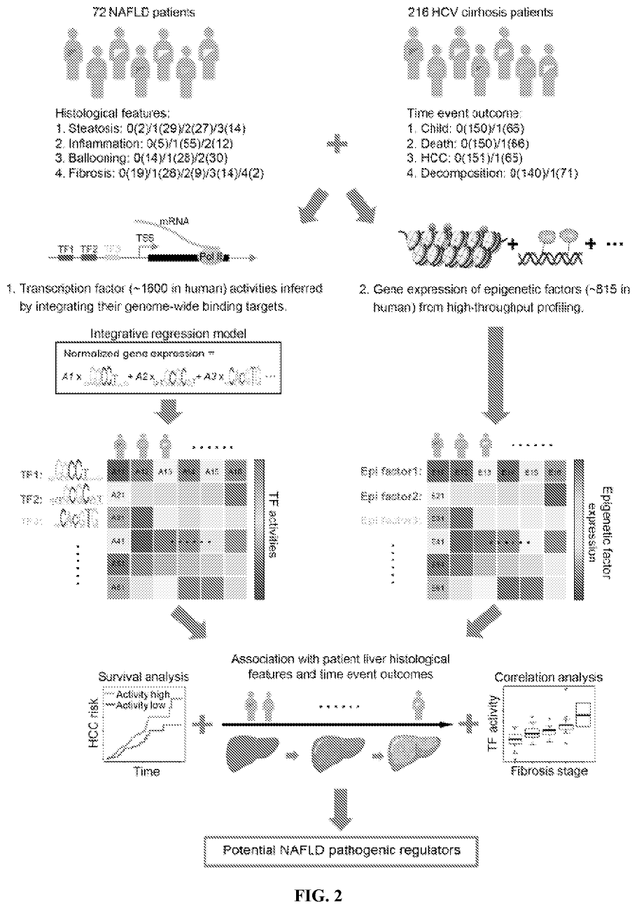

NASH using MOSAICS. It was reasoned that transcription or epigenetic factors might have the strongest impact on a broad array of pathways, human gene expression data were analyzed to nominate candidate factors whose activities were either increased or decreased chronic liver disease (FIG. 2). Putative transcription factor activities were modeled using gene expression data from NASH (72 patients; GSE130970, previous study) and HCV cirrhosis cohorts (216 patients; GSE15654, previous study). Since transcription factor gene expression levels did not always reflect their functional importance, those with the highest or lowest activities were chose based on the induction or suppression of their downstream targets (See methods for details). In contrast, the epigenetic genes were ranked only based on their differential expression in human cirrhosis samples. To further narrow down the most influential factors, candidate genes were also associated with histologic features such as fibrosis, inflammation, ballooning, and steatosis, as well as temporal events such as Child-Pugh score, liver decompensation, cancer development, and death. Using this clinico-genomic pipeline, 217 transcriptional and 255 epigenetic regulators were identified that have the highest likelihood of having an impact in human NASH.

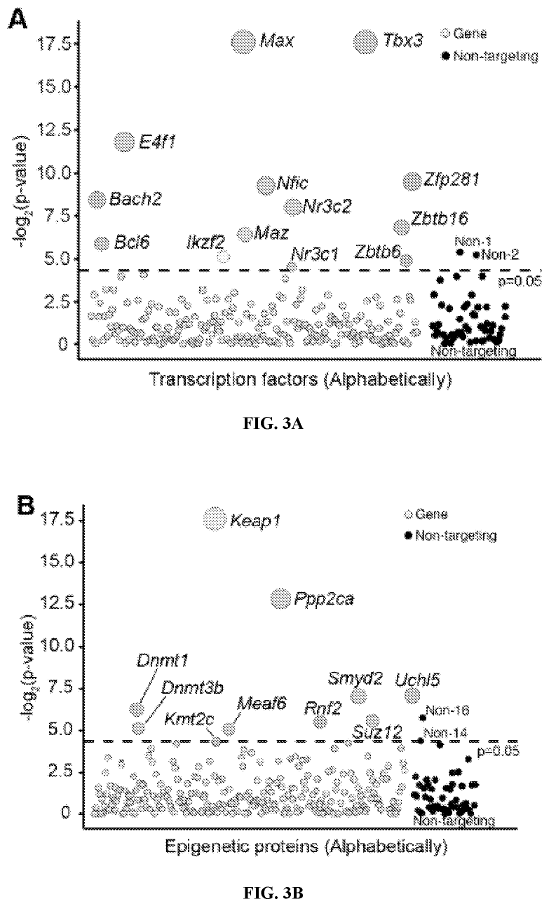

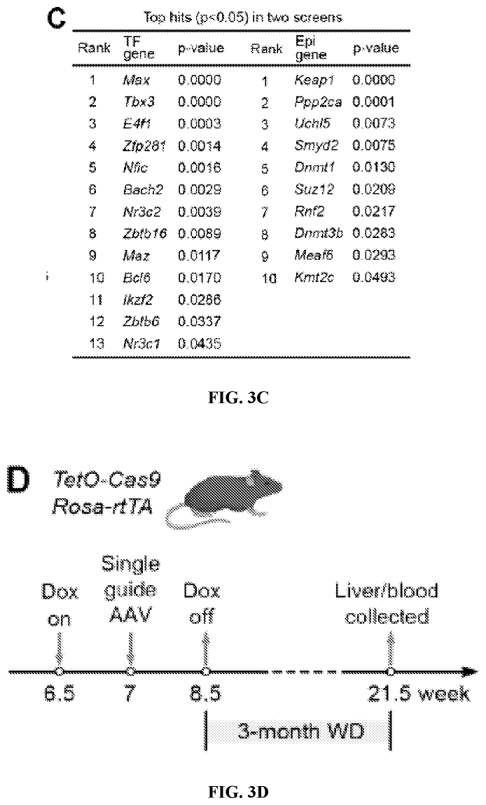

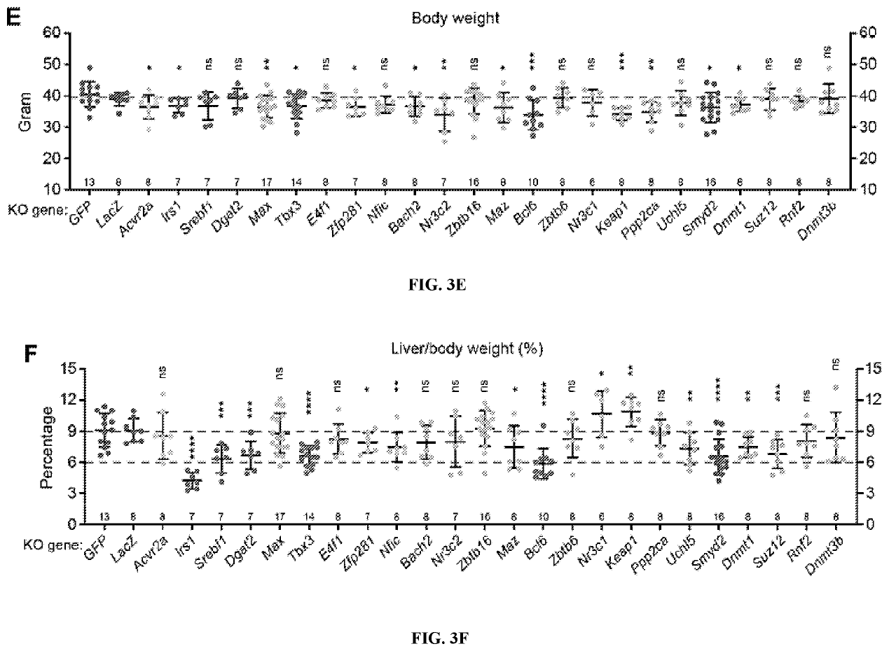

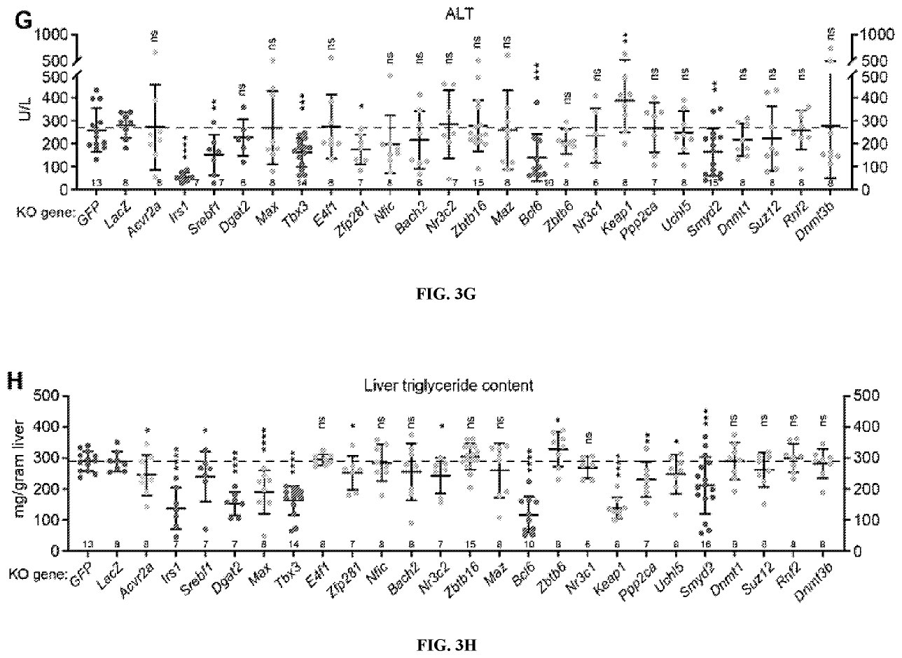

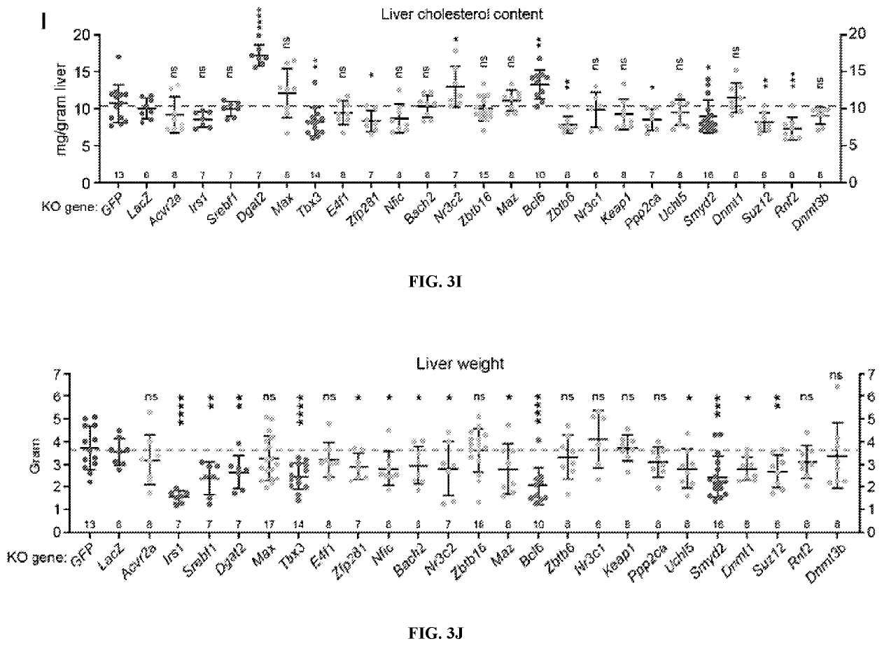

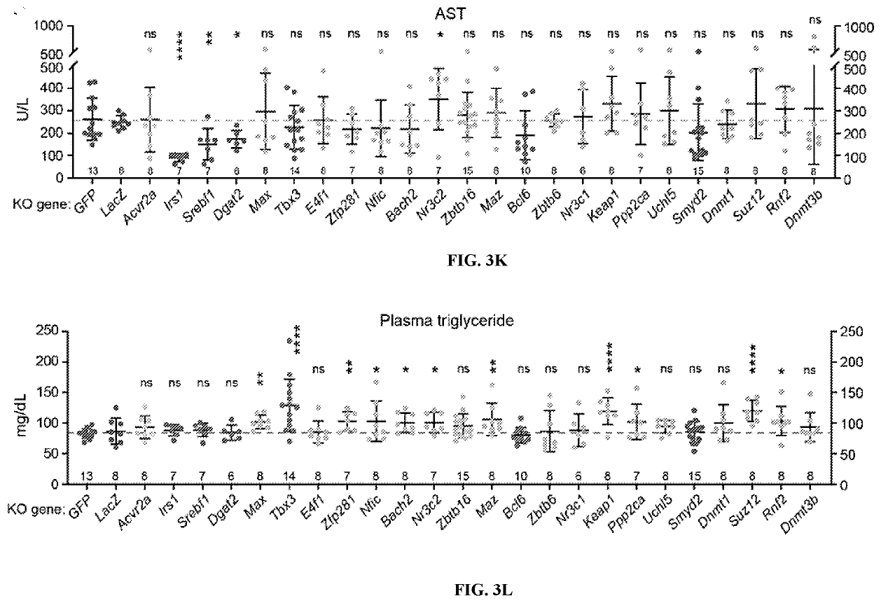

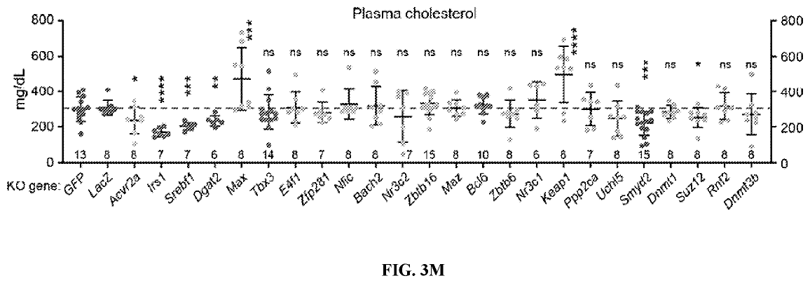

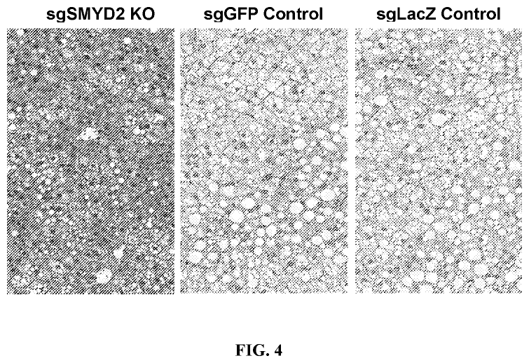

Next, sgRNA libraries were created corresponding to these two gene sets (transcription and epigenetic factors), and were used to generate somatically mutated mouse livers using the MOSAICS platform (FIG. 1). After 6 months of chow or WD, the sgRNAs associated with the most clonal expansion in WD livers targeted 13 and 10 genes in each screen, respectively (FIGS. 3A-3B). Most of the top genes had no known connection with liver disease or NASH.

### Establishment of Conditional Deletion Methods for Rapid Liver Disease Phenotyping

To further ascertain which of the top genes, when genetically ablated in all hepatocytes, have the most positive influences on metabolism under WD conditions, rapid conditional KO approaches were developed. The most enriched sgRNAs were cloned for each of the top genes into the MOSAICS vector, then delivered saturating doses of these AAVs with individual guides into Cas9 expressing mice to achieve single-gene whole-liver deletion. This system was first tested using the four known NASH genes that scored as the most positively selected hits from the 63 NASH gene screen. High titers of MOSAICS AAV8-sgRNAs against GFP (control 1), LacZ (control 2), Smyd2 were injected into Cas9 expressing mice at 6.5 weeks of age, then were fed WD for 3 months. After 3 months of WD, each conditional CRISPR KO mouse was phenotyped for body weight, liver weight, liver lipids (triglyceride, cholesterol), plasma parameters (ALT, AST, triglyceride, cholesterol) and histology. Furthermore, the protection against fatty liver disease was evident from improved plasma parameters, decreased liver lipid content (FIGS. 3D-3K), and reduced steatosis (FIG. 3L). These findings were tightly correlated with the degree of liver/body weight ratio reduction, a surrogate marker of fat accumulation and NASH. These data showed that CRISPR mediated liver-wide conditional deletion would be an effective and rapid method of organ level validation.

### Deletion of Smyd2 Showed Protection Against Fatty Liver Disease

Deletion of the epigenetic factor Smyd2, showed significant reductions in body weights (FIG. 3E) and the most substantial reductions in liver/body weight ratios compared to controls after 3 months of WD (dark dots in FIG. 3F). SMYD2, has not been previously studied in fatty liver disease. This KO model showed the most significant reductions in liver injury as measured by ALT (FIG. 3G), a trend toward reduced AST (FIG. 3K), and the most significant reductions in liver triglyceride levels (FIG. 3H). We also observed reduced plasma cholesterol in Smyd2 KO mice (FIG. 3I). Importantly, a clear reduction was observed in hepatic steatosis in Smyd2 KO livers compared with controls and most other KO models. The phenotypes of Smyd2 KO mice were comparable to Srebf1 and Dgat2 KO mice, suggesting potent regulation of lipid metabolism by this gene.

### Transcriptional Analysis Revealed Diverse Mechanisms of Lipid Regulation

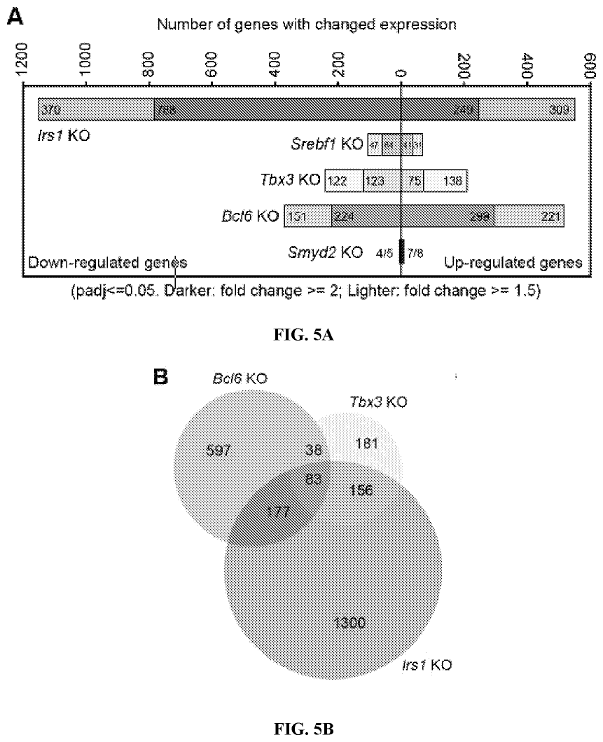

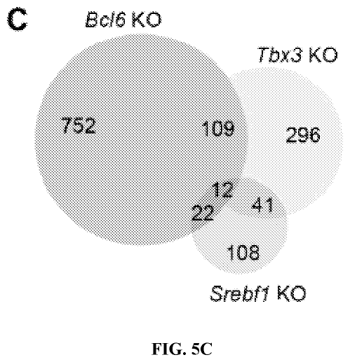

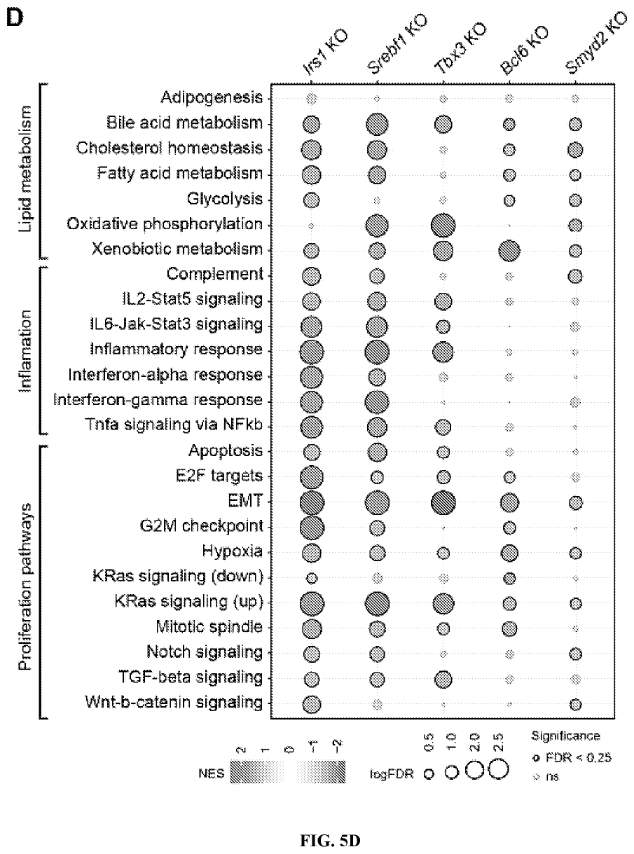

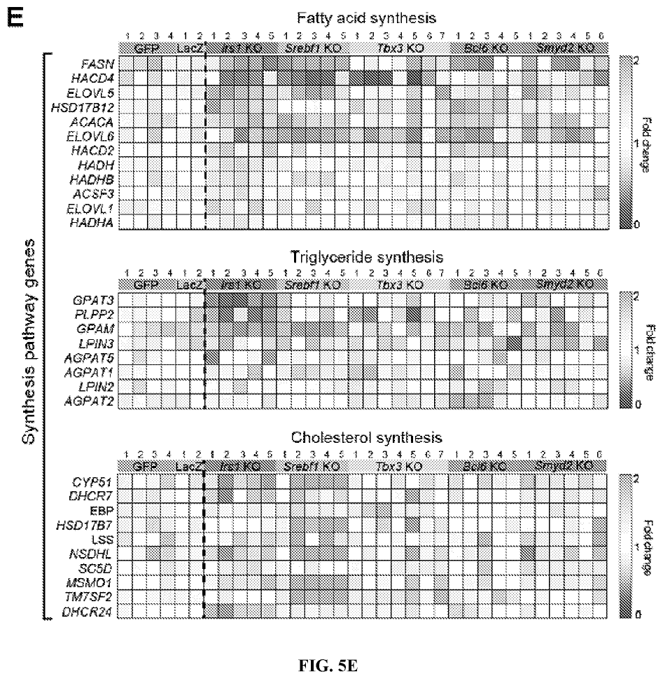

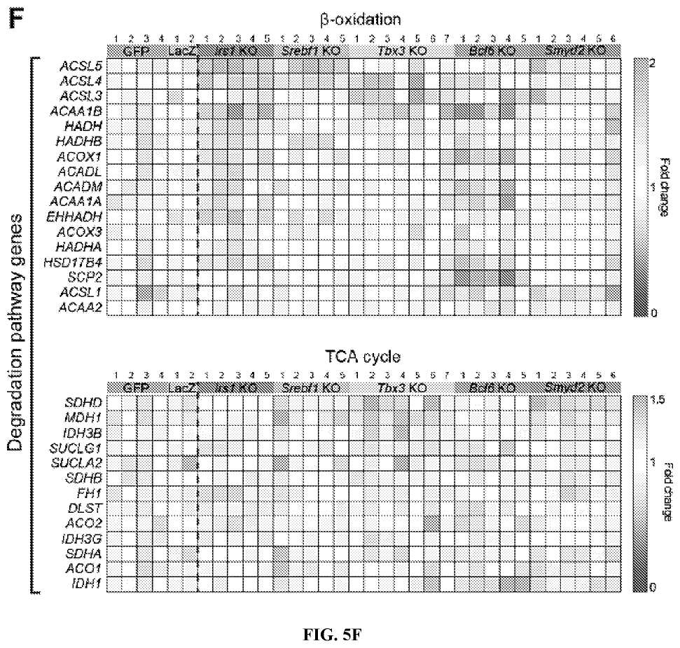

To investigate the gene expression changes in WD treated livers carrying fitness promoting mutations, RNA-seq were performed on control (sgGFP, sgLacZ) and Smyd2 KO livers that were generated with saturating doses of MOSAICS AAVs. Smyd2 KO livers showed the smallest number of genes with expression changes as compared to Irs1, Tbx3, and Bcl6 KO livers (FIG. 5A). We further examined the expression of genes involved in lipogenesis and fatty acid oxidation. Deletion of Smyd2 led to decreased expression of fatty acid and triglyceride synthesis genes (FIG. 5B). Upregulation of many TCA cycle genes in Smyd2 KO livers (FIG. 5C lower panel) was observed, suggesting that these livers have higher rates of acetyl-CoA consumption. These data indicate shared and unique mechanisms by which mutant hepatocytes converge on decreased steatosis.

### A Selective Small Molecule Inhibitor of SMYD2 Can Prevent Fatty Liver Disease

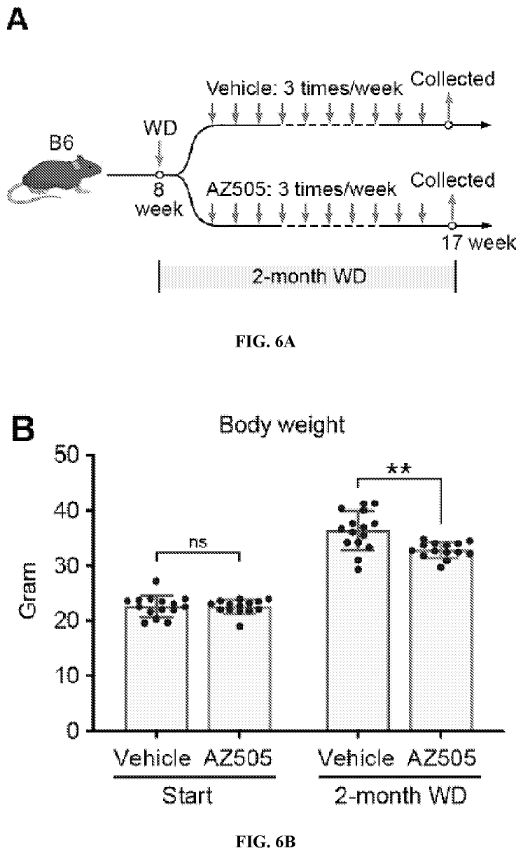

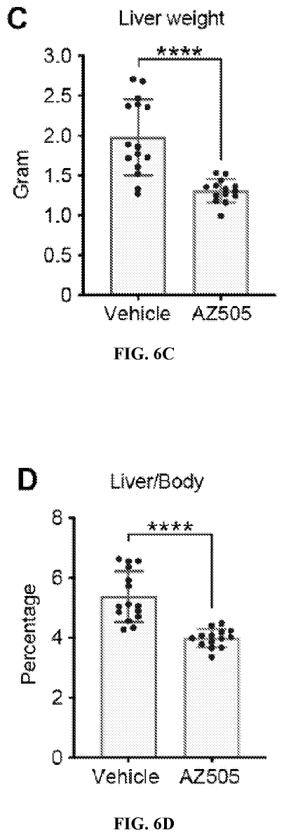

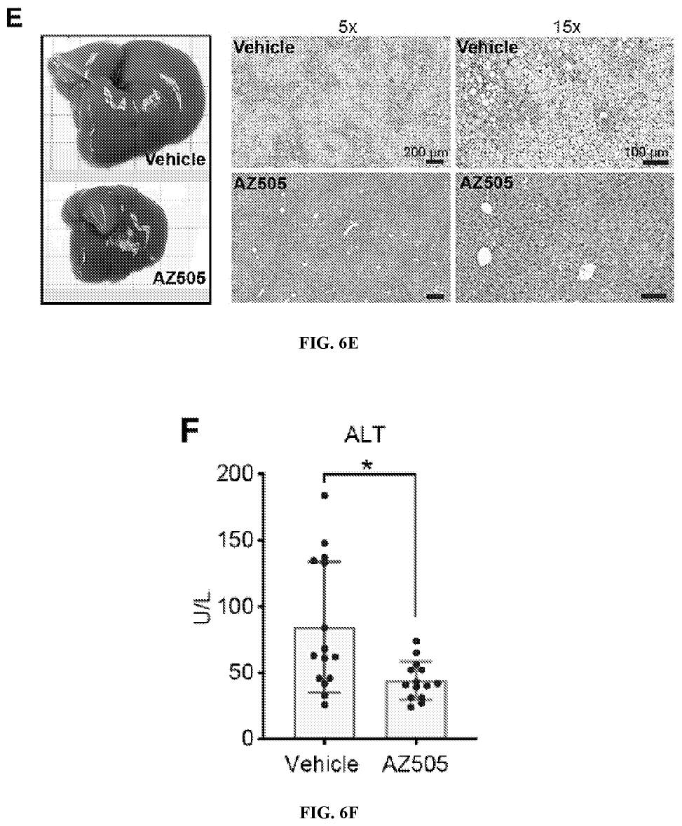

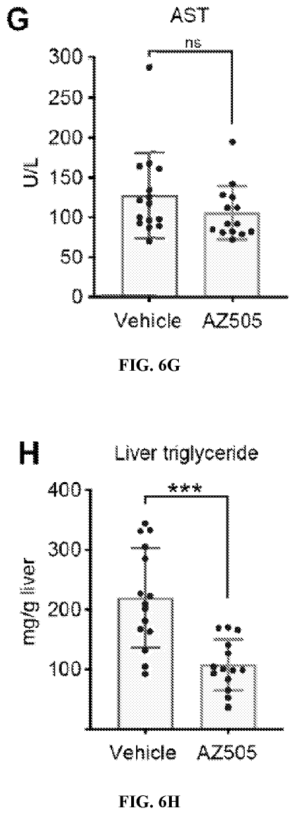

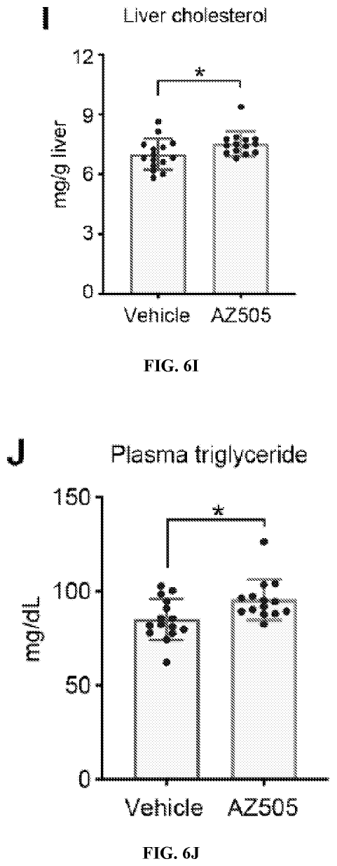

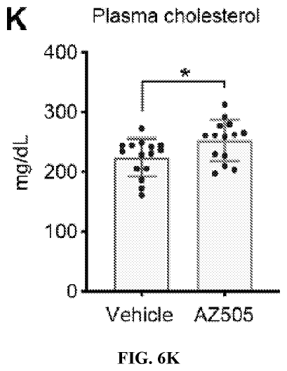

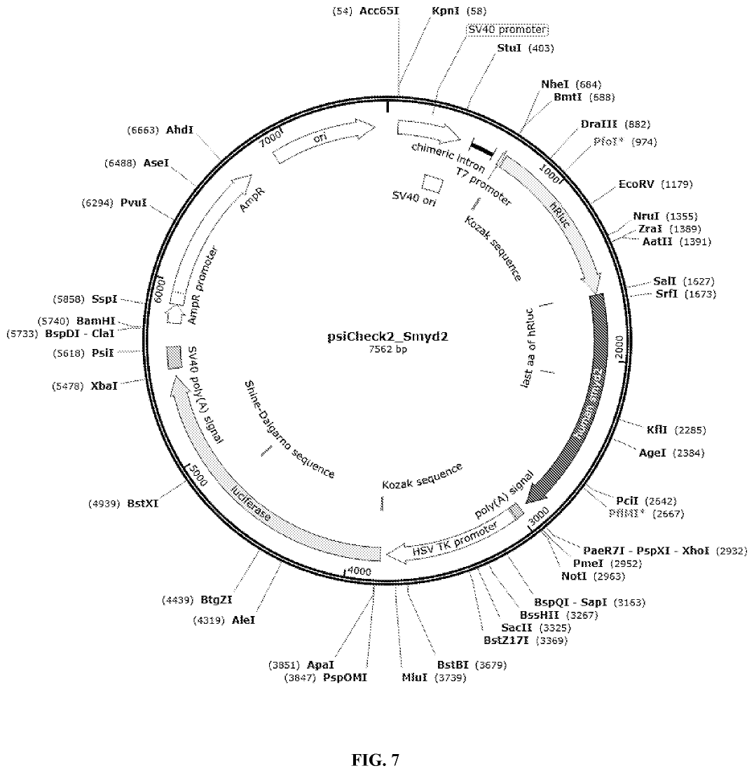

In order to determine if chemical SMYD2 inhibition could serve as orthogonal validation of the genetic findings, and to investigate the therapeutic potential of SMYD2 blockade in fatty liver disease, AZ505 was used. AZ505 is a selective SMYD2 inhibitor with an IC50 of 0.12 μM that has strong selectivity for SMYD2 (>600 fold selectivity). WD was fed to WT mice at 8 weeks of age, followed by treatment with vehicle control or AZ505 with a dose of 10 mg/kg (intraperitoneal injection) three times per week (FIG. 6A). This dose was well tolerated in the present experiment. Two months after continued WD and inhibitor treatment, it was observed significant reductions in body weight, liver weight, and liver/body weight ratios in AZ505 vs. vehicle treated mice (FIG. 6B-6D). Smaller, less pale livers were observed in AZ505 treated mice, and liver histology showed dramatically decreased steatosis in contrast to a wide range of macro and microscopic lipid droplet deposition in vehicle treated livers (FIG. 6E). Accordingly, AZ505 treated mice showed improved transaminitis (FIG. 6F,G) and a significant decrease in liver triglycerides (FIG. 6H), whereas the liver cholesterol (FIG. 6I) and plasma lipids (FIG. 6J, 6K) showed a modest increase. Thus, a small molecule SMYD2 inhibitor can recapitulate the phenotypes of SMYD2 CRISPR KO mice. Because AZ505 binds to the active center of SMYD2, these experiments also showed that mechanistically, SMYD2 enzymatic activity, and not just protein levels, contributed to fatty liver phenotypes. In summary, several lines of orthogonal evidence suggest that SMYD2 is a promising NASH target.

### Example 3. Design and Testing of SMYD2 siRNA and Inhibition of SMYD2 mRNA Expression

Specific siRNA sequences were designed to target human SMYD2 for the treatment of NASH. Based on what we have learned about the biology of SMYD2 in mouse models, SMYD2 represents a promising therapeutic target for human NASH.

Tests identified optimized siRNA sequences corresponding to the human sequences of human SMYD2. This involved screening many candidate siRNAs per gene target using in vitro luciferase reporter-based assays. In brief, full length SMYD2 cDNA was cloned into a psicheck2 plasmid backbone containing a luciferase gene. This created a Renilla luciferase-SMYD2 fusion gene. The luciferase assay is a dual reporter system with Firefly luciferase as a control and Renilla luciferase as the read out for transcription and translation. Thus, the ratio of Renilla to Firefly signal accounts for variations in transfection efficiency and cell viability. The assay was carried out in a 96 well plate format with technical replicates using a 48 hour time point for the assay readout. The reporter along with siRNAs were co-transfected into

Cos7 monkey kidney cells using lipofectamine. After 48 hours, the cells were lysed and the signal was captured by a luminometer using the substrates for Firefly and Renilla. Effective siRNAs against a target gene showed a reduced ratio for Renilla vs. Firefly signal compared to non-targeting controls. Optimized siRNAs will be modified by conjugating to acetylgalactosamine (GaINAC) in the standard fashion and tested in vivo.

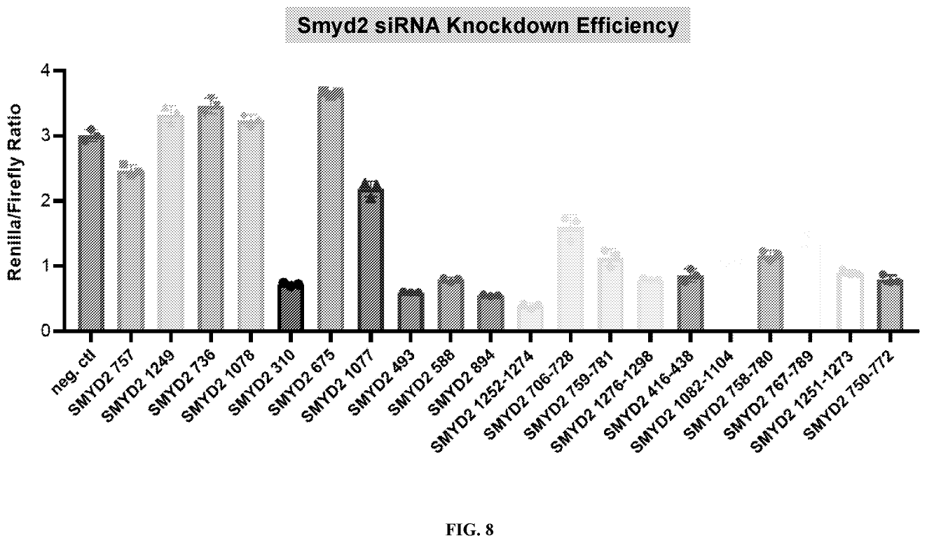

The human SMYD2 luciferase reporter plasmid construct was designed (FIG. 8 and Table 2, and the inserted sequence is listed as Seq ID No.: 173) and used for siRNA testing in Cos7 cells. FIG. 8 and Table 3 provided efficacy of Seq ID No.: 1-20 siRNAs shown in Table 1. (Note: The sequences listed in Table 3 include two nucleotide overhangs at the C-terminal.) The results showed that it was possible to block SMYD2 expression through siRNA inhibitors, some of which were able to reduce the expression to more than 70%, such as Seq ID No.: 8 and 10-11. These results proved siRNA inhibitors were capable of blocking SMYD2 expression, and in turn could reverse pathological changes leading to liver diseases, such as NASH and NAFLD.

TABLE 2
Plasmid Construct

| | |
|---|---|
| Target: | human Smyd2, |
| | RefSeq: NM_020197.3 |
| clone: | Smyd2 with a point mutation |
| Dual-Luc Assay | |
| notes: | |
| Cells: | Cos7 cells |
| Luciferase Vector: | psiCheck2 |
| Insert Length: | 1299 bp (3′-UTR not included) |
| Insert Sequence: | AGGGCCGAGGGCCTCGGCGGCCTGGAGCGC |
| | TTCTGCAGCCCGGGCAAAGGCCGGGGGCTG |
| | CGGGCTCTGCAGCCCTTCCAGGTGGGGGAC |
| | TTGCTGTTCTCCTGCCCGGCCTATGCCTAC |
| | GTGCTCACGGTCAACGAGCGGGGCAACCAC |
| | TGCGAGTACTGCTTCACCAGGAAAGAAGGA |
| | TTGTCCAAATGTGGAAGATGCAAGCAGGCA |
| | TTTTACTGCAATGTGGAGTGTCAGAAAGAA |
| | GATTGGCCCATGCACAAGCTGGAATGTTCT |
| | CCCATGGTTGTTTTTGGGGAAAACTGGAAT |
| | CCCTCGGAGACTGTAAGACTAACAGCAAGG |
| | ATTCTGGCCAAACAGAAAATCCACCCAGAG |
| | AGAACACCTTCGGAAAAATTGTTAGCTGTG |
| | AAGGAGTTTGAATCACATCTGGATAAGTTA |
| | GACAATGAGAAGAAGGATTTGATTCAGAGT |
| | GACATAGCTGCTCTCCATCACTTTTACTCC |
| | AAGCATCTCGGATTCCCTGACAATGATAGC |
| | CTCGTAGTACTCTTTGCACAGGTTAACTGT |
| | AATGGCTTCACAATTGAAGATGAAGAACTT |
| | TCTCATTTGGGATCAGCGATATTTCCTGAT |
| | GTTGCATTGATGAATCATAGCTGTTGCCCC |
| | AATGTCATTGTGACCTACAAAGGGACCCTG |
| | GCAGAAGTCAGAGCTGTACAGGAAATCAAG |
| | CCGGGAGAGGAGGTTTTTACCAGCTATATT |
| | GATCTCCTGTACCCAACGGAAGATAGAAAT |
| | GACCGGTTAAGAGATTCTTATTTCTTTACC |
| | TGTGAGTGCCAGGAGTGTACCACCAAGGAC |
| | AAGGATAAGGCCAAGGTGGAAATCCGGAAG |
| | CTCAGCGATCCCCCAAAGGCAGAAGCCATC |
| | CGAGACATGGTCAGATATGCACGCAACGTC |
| | ATTGAAGAGTTCCGGAGGGCCAAGCACTAT |
| | AAATCCCCTAGTGAGCTGCTGGAGATCTGC |
| | GAGCTCAGCCAGGAGAAGATGAGCTCTGTG |
| | TTTGAGGACAGTAACGTGTACATGTTGCAC |
| | ATGATGTACCAGGCCATGGGTGTCTGCTTG |
| | TACATGCAGGACTGGGAAGGAGCCCTGCAA |
| | TATGGACAGAAAATCATTAAGCCCTACAGT |
| | AAGCACTATCCTTTGTACTCCCTCAACGTG |
| | GCCTCCATGTGGTTGAAGCTAGGGAGACTC |
| | TACATGGGCCTGGAACACAAAGCCGCAGGG |
| | GAGAAAGCCCTGAAGAAGGCCATTGCAATC |
| | ATGGAAGTAGCTCACGGCAAAGATCATCCA |
| | TATATTTCTGAGATCAAACAGGAAATTGAA |
| | AGCCACTGA |
| Timepoint: | Luciferase reporter and |
| | siRNA co-transfected, 24 h, |
| | triplicates |

TABLE 3
Human SMYD2 siRNA Knockdown Efficiency

| | | % mRNA | |
|---|---|---|---|
| | | remaining | |
| Seq | | (compared to | |
| ID | SIRNA ID | Ctrl siRNA) | Sense siRNA Sequence |
| 2 | SMYD2 757 | 82.3 | CCGGUUAAGAGAUUCUUAUUU |
| 3 | SMYD2 1249 | 110.9 | CGGCAAAGAUCAUCCAUAUUU |
| 4 | SMYD2 736 | 115.1 | CCCAACGGAAGAUAGAAAUUU |
| 5 | SMYD2 1078 | 107.5 | CCUGCAAUAUGGACAGAAAUU |
| 6 | SMYD2 310 | 24.0 | GGAGACUGUAAGACUAACAUU |
| 7 | SMYD2 675 | 121.4 | GAGCUGUACAGGAAAUCAAUU |
| 8 | SMYD2 1077 | 72.7 | CCCUGCAAUAUGGACAGAAUU |
| 9 | SMYD2 493 | 20.0 | CGGAUUCCCUGACAAUGAUUU |
| 10 | SMYDS 588 | 26.3 | CAGCGAUAUUUCCUGAUGUUU |
| 11 | SMYD2 894 | 18.3 | CACGCAACGUCAUUGAAGAUU |
| 12 | SMYD2 1252- | 13.1 | CAAAGAUCAUCCAUAUAUUUC |
| | 1274 | | |
| 13 | SMYD2 706- | 53.2 | GGUUUUUACCAGCUAUAUUGA |
| | 728 | | |
| 14 | SMYD2 759- | 37.7 | GGUUAAGAGAUUCUUAUUUCU |
| | 781 | | |
| 15 | SMYD2 1276- | 26.6 | GAUCAAACAGGAAAUUGAAAG |
| | 1298 | | |
| 16 | SMYD2 416- | 28.5 | GAUAAGUUAGACAAUGAGAAG |
| | 438 | | |
| 17 | SMYD2 1082- | 35.2 | CAAUAUGGACAGAAAAUCAUU |
| | 1104 | | |
| 18 | SMYD2 758- | 38.8 | CGGUUAAGAGAUUCUUAUUUC |
| | 780 | | |
| 19 | SMYD2 767- | 46.4 | GAUUCUUAUUUCUUUACCUUU |
| | 789 | | |
| 20 | SMYD2 1251- | 30.0 | GCAAAGAUCAUCCAUAUAUUU |
| | 1273 | | |
| 21 | SMYD2 750- | 26.5 | GAAAUGACCGGUUAAGAGAUU |
| | 772 | | |

### Example 4

Some of these human siRNAs against SMYD2 will be tested in human cell line models of NASH. The tests will be performed using optimized siRNAs and/or their conjugates with acetylgalactosamine (GalNAc) to improve liver targeting. The experimental testing of candidate siRNAs has three steps. First, we use the above identified siRNAs in human liver cancer cells, such as Huh7 or HepG2, to knockdown target genes of interest, e.g., SMYD2. Specifically, Huh7 cells are grown to 60-80% confluence and then transfected with siRNAs. Opti-MEM (Thermo Fisher 31985062) containing the siRNAs and Lipofectamine are combined, incubated, and then added to cells for transfection (400 μl/well to a 6-well plate or 2.4 ml to a 100 mm plate). Transfection medium is replaced after 6 h with medium containing 100 μM FA. Then, we will feed cells lipids and label with lipid dyes. Lastly, we will perform fluorescence-activated cell sorting (FACS) to quantitate lipid accumulation. FACS to purify specific cell populations based on phenotypes detected by flow cytometry. This method enables characterization of a single cell population without the influence of other cells. Cells are stained with 1 ml PBS containing 10 μg of BODIPY® 493/503 (BD) (Thermo Fisher D3922) for 15 min at room temperature, then subject to FACs measurements. Early studies show that in lipophilic fluorophore stained cells, fluorescence intensity measured by FC reflects lipid levels (Wolins, 2018).

An aliquot of the cells grown under each condition is assayed for triacylglycerol (TG). In brief, cells are trypsinized, washed once with PBS, resuspended in PBS with 10 mM EDTA, and then counted. Cells are divided in triplicate into 13×100 mm glass tubes in a final volume of 200 μl. Triolein standards (Sigma T7140) are also prepared in a final volume of 200 μl PBS/10 mM EDTA in 13×100 mm glass tubes. TGs are extracted and quantified. Briefly, 2 ml of isopropanol: hexane: water (40:10:1) is added to cells or standards and the samples are vortexed, covered, and incubated at room temperature for 30 min. Then, 500 μl of a 1:1 mixture of hexane: diethylether is added to the samples followed by vortexing and incubating for an additional 10 min at room temperature. Next, 1 ml of water is added to samples, tubes are vortexed, and layers are allowed to separate at room temperature while covered for 30-45 min. Using Pasteur pipettes, the top layer is transferred to 12×75 mm glass tubes and dried under N₂ to completion. Following the drying step, 400 μl of Infinity triglyceride reagent (Thermo Scientific TR22421) is added to each tube and vortexed. Tubes are covered and incubated for 90 min at 37° C. with shaking at 250 rpm. Finally, 300 μl of each sample is transferred to 96-well plates and absorbance is measured at 540 nm using a microplate reader. To determine whether fixing cells affected the TG measurement, TG is quantified in fixed and unfixed cells from the same batch of Huh7 cells incubated in 2 mM FA.

### Example 5. Methods and Procedures

The present disclosure used the following methods and procedures.

### Mouse Strains and Breeding

All mice were handled in accordance with the guidelines of the Institutional Animal Care and Use Committee at UT Southwestern. All experiments were done in an age and sex controlled fashion unless otherwise noted. C57BL/6 strain background mice were used for all the experiments. LSL-tdTomato (strain #007914) and Rosa-rtTA; TetO-Cas9 mice (#029415) were obtained from The Jackson Laboratory. Mice homozygous for both Rosa-rtTA and TetO-Cas9 were used to ensure a high Cas9 expression level in the liver. Western Diet (WD) used for NASH modeling is described in (Tsuchida et al., 2018). It is composed of high fat solid food (ENVIGO #TD.120528) and high sugar water containing 23.1 g/L d-fructose (Sigma-Aldrich #F0127) and 18.9 g/L d-glucose (Sigma-Aldrich #G8270).

### Fluorescent Imaging and Image Processing

For fluorescent imaging, liver pieces were fixed in buffered formalin (Fisherbrand #245-685) for 24 h with gentle shaking at 4° C. and then transferred into 30% sucrose (w/v) solution for another 24 h with shaking at 4° C. The livers were then embedded and frozen in Cryo-Gel (Leica #39475237), and sectioned at a thickness of 16 μm. Images were taken using a Zeiss Axionscan Z1 system in the UTSW Whole Brain Microscopy Facility to visualize and quantify Tomato clones. To statistically analyze the percentage of Tomato positive cells, black and white fluorescent images were taken from the same slide using an Olympus IX83 microscope at 4× magnification. Two different fields were taken for each liver.

The percentage of Tomato positive cells (bright areas) was analyzed using ImageJ.

### H&E, Immunohistochemistry (IHC) and TUNEL Staining

Liver pieces were fixed in buffered formalin (Fisherbrand #245-685) for 24 h with gentle shaking at 4° C. and then transferred to 70% EtOH for another 24 h with shaking at 4° C. Paraffin embedding, liver sectioning (4 μm thickness), and H&E staining were performed at the UT Southwestern Tissue Management Shared Resource Core. For IHC staining, the following primary antibodies were used: Pten (CST #9559, IHC 1:200); Ki67 (Abcam #AB15580, IHC 1:200). IHC). IHC was performed as previously described (Zhu et al., 2010). TUNEL staining was performed on paraffin embedded liver sections using In Situ Cell Death Detection Kit, Fluorescein (Roche #C755B40) according to the manufacturer's protocol. QuPath software (https://qupath.github.io/) was used to quantify Ki67 and TUNEL staining.

### Plasma Parameters and Liver Lipid Measurements

Blood samples were taken using heparinized tubes from the inferior vena cava immediately after sacrificing the mouse, and then transferred into 1.5 ml tubes and centrifuged at 2000 g for 15 min at 4° C. The supernatant after centrifugation (plasma) was analyzed for AST, ALT, cholesterol, and triglyceride using VITROS MicroSlide Technology at the UT Southwestern Metabolic Phenotyping Core. 100-150 mg of liver per sample was weighted and used for lipid extraction and quantification at the UT Southwestern Metabolic Phenotyping Core.

1. A small interfering RNA (siRNA) molecule capable of downregulating gene expression of SMYD2 (SET and Myeloid-Nervy-DEAF-1 domain-containing protein 2) or a variant thereof.

2. The siRNA molecule of claim 1, wherein the siRNA molecule comprises a nucleotide sequence that is 2 to 30 nucleotides in length and is at least 80% homologous to at least 2 to 30 contiguous nucleotides of a human SMYD2 cDNA sequence.

3. (canceled)

4. The siRNA molecule of claim 2, wherein the siRNA molecule comprises at least one nucleotide sequence that is 2 to 30 nucleotides in length and is at least 80% homologous to at least one of the cDNA sequences of SEQ ID Nos.: 2-58, or wherein the siRNA molecule comprises at least one nucleotide sequence that is 2 to 30 nucleotides in length and is at least 80% homologous to at least one of the cDNA sequences of SEQ ID Nos.: 9, 11, or 12.

5. (canceled)

6. The siRNA molecule of claim 1, wherein the siRNA molecule targets the open reading frame or the 5′ or 3′ UTRs of the human SMYD2 gene.

7. The siRNA molecule of claim 2, wherein the siRNA molecule comprises a nucleotide sequence of SEQ ID Nos: 59-172, a nucleotide sequence having at least 80% identity to any one of SEQ ID NOs: 59-172, or any combination thereof.

8. The siRNA molecule of claim 7, wherein the siRNA molecule comprises a nucleotide sequence having at least 90% identity of any one of Seq ID Nos.: 66, 68, 69, 123, 125, 126, or any combination thereof.

9. The siRNA molecule of claim 1, wherein the siRNA molecule comprises at least one sense sequence, at least one antisense sequence, or both a sense and an antisense sequence.

10. The siRNA molecule of claim 9,
wherein the at least one sense sequence comprises a nucleotide sequence of any one of SEQ ID NOs.: 59-115; and/or
wherein the at least one antisense sequence comprises a nucleotide sequence of any one of SEQ ID NOs.: 116-172.

11. (canceled)

12. The siRNA molecule of claim 1, wherein the siRNA molecule downregulates at least about 30%, at least about 40%, at least about 50%, at least about 60%, at least about 70%, at least about 80%, or at least about 90% of gene expression of the human SMYD2 or a variant thereof associated with a liver disease.

13-14. (canceled)

15. The siRNA molecule of claim 12, wherein the liver disease comprises fatty liver disease (FLD), alcohol-related liver disease (ARLD), non-alcoholic fatty liver disease (NAFLD), non-alcoholic steatohepatitis (NASH), cirrhosis from any etiology, liver cancer, or any combination thereof.

16. The siRNA molecule of claim 1, wherein the siRNA molecule is conjugated to least one liver targeting ligand or the siRNA molecule comprises at least one chemical modification.

17. (canceled)

18. The siRNA molecule of claim 17,
wherein the liver targeting ligand comprises at least one N-Acetylgalactosamine (GalNAc) conjugate; or
wherein the chemical modification comprises at least one ribosugar moiety of its nucleotide sequence.

19-21. (canceled)

22. The siRNA molecule of claim 18, wherein the at least one ribosugar moiety is modified with 2 2′-O-methyl (2′OMe), 2′-deoxy-2′-fluoro (2′F), 2′-deoxy, 5-C-methyl, 2′-O-(2-methoxyethyl) (MOE), 4′-thio, 2′-amino, 2′-C-allyl, or any combination thereof.

23. (canceled)

24. A pharmaceutical composition comprising the siRNA of claim 1, and at least one excipient.

25. (canceled)

26. The pharmaceutical composition of claim being a nanoparticle or a viscous formulation.

27. (canceled)

28. A method of treating a subject in need thereof, the method comprising administrating a therapeutically effective amount of the siRNA of claim 1 to the subject.

29. The method of claim 28, wherein the subject in need thereof, is a human subject having or suspected of having a liver disease selected from fatty liver disease (FLD), alcohol-related liver disease (ARLD), non-alcoholic fatty liver disease (NAFLD), nonalcoholic steatohepatitis (NASH), cirrhosis, liver cancer, and any combination thereof.

30-31. (canceled)

32. The method of claim 28, wherein the method of administering comprises parenteral administration.

33. The method of claim 28, wherein the therapeutically effective amount is the amount effective to
(i) increase life expectancy of the subject;
(ii) improve liver function of the subject;
(iii) attenuates liver fibrosis in the subject; or
(iv) prevents additional liver fibrosis in the subject,
when compared to an untreated subject with identical disease condition and predicted outcome.

34-36. (canceled)

37. A kit comprising:
a. a container holding the siRNA of claim 1 or a composition thereof;
b. a pharmaceutical administrative means; and
c. an instruction of use.
